(19) United States
(12) Patent Application Publication
(10) Pub. No.: US 2024/0027464 A1
(43) Pub. Date: Jan. 25, 2024

(54) FLUORESCENCE ASSAY FOR IDENTIFYING PATHOGENS IN A SAMPLE, AND COMPUTER-IMPLEMENTED SYSTEMS FOR CARRYING OUT SUCH ASSAYS

(71) Applicant: Oxford Nanoimaging Limited, Oxford, oxfordshire (GB)

(72) Inventors: James H. Felce, Oxford (GB); Yaoyao Xiong, Oxford (GB); Bo Jing, Oxford (GB); Anusuya Banerjee, Oxford (GB); Beata Klejevskaja, Oxford (GB)

(73) Assignee: Oxford Nanoimaging Limited, Oxford, Oxfrodshire (GB)

(21) Appl. No.: 18/035,454

(22) PCT Filed: Nov. 5, 2021
(86) PCT No.: PCT/EP2021/080780

§ 371 Date: May 4, 2023

(30) Foreign Application Priority Data
Nov. 5, 2020 (GB) 2017526.1

(51) Int. Cl. G01N 33/58; G01N 21/64; G16H 50/80

(52) U.S. Cl. CPC G01N 33/582; G01N 21/6458; G16H 50/80; G01N 2333/165

(57) ABSTRACT

The present application discloses a method of identifying and characterising intact pathogens in a sample using a fluorescence imaging system, in particular for characterising bacteria and viruses, and associated systems and kits for implementing the imethod. The method involves incubating a sample with fluorescent probes from at least two of the following categories: a fluorescent probe for binding to a pathogen surface carbohydrate; a nucleic acid stain; and a membrane stain. The sample is then imaged using fluorescence imaging apparatus to detect candidate objects, and the fluorescence characteristics of the candidate objects used to identify whether the object is a pathogen and, if so, the type of pathogen. Particularly preferred implementations employ flow to improve data acquisition, and machine learning classification algorithms to distinguish different pathogen types.

### FIELD OF THE INVENTION

The present invention relates to methods for detecting and identifying pathogens by fluorescence microscopy, in particular methods for rapidly identifying intact viruses and bacteria in bodily fluids with minimal sample preparation. The invention also relates to a kit of parts and a computer implemented system for carrying out such methods.

### BACKGROUND

Established methods to detect pathogens in bodily fluids are (isothermal) PCR to detect genomic material, detecting antigens from the pathogen via antibodies or aptamers, or detecting human antibodies against antigens that indirectly indicate the presence of pathogens. These methods output easily interpretable, one-dimensional data points, often indicating the number of pathogen components present in the sample. However, diagnostic tests based on these methods are only sensitive to one type of pathogen, take hours to run, suffer from false negative and false positive issues, are insensitive, and/or are costly due to the use of difficult to manufacture reagents like enzymes and antibodies.

It is also known to use microscopy to identify pathogens, but the existing techniques suffer from a number of limitations.

For example, conventional microscopy-based detection methods can only pick up large pathogens (greater than 1 μm in size) and the sensitivity is low, usually requiring a high pathogen concentration to be present, or culturing of the pathogen before imaging. Furthermore, even for pathogens of a sufficient size to be effectively imaged, these techniques are generally limited to distinguishing between broad classes of pathogens (for example, gram-positive vs gram-negative), without being able to characterise the pathogens within those classes, such as the specific species of pathogen.

Fluorescence-based techniques are also known which detect specific markers on a given pathogen using fluorescently labelled molecules specific to that marker, such as antibodies or aptamers. However, whilst these techniques are good for identifying whether a specific pathogen type is present in a sample, they are poorly suited to broad tests which examine samples for a spectrum of pathogen types. In particular, it is impractical and expensive to introduce and distinguish between multiple different types of fluorescently labelled antibodies/aptamers in a sample. Furthermore, the method will generally be incapable of identifying new pathogen forms, against which specific binding molecules have not yet been developed.

As an example of a fluorescence-based method for detecting pathogens, WO 2020/089621 describes a method of functionalising virions with negatively charged polymers (in particular, a polynucleotide) by treating the viruses with a polyvalent cation, and then adding the negatively charged polymer. The authors describe the possibility of immobilising the virions to a substrate, and then labelling with a virus-specific identification agent (for example, a strain-specific antibody, aptamer or complementary genome probe). The identification agent and, optionally, the polymer, are labelled with detectable labels, such as fluorophores, to allow their detection by single molecule microscopy.

More recent work reported in Shiaelis et al., describes using the same cation-mediated functionalisation of virions with short fluorescent DNAs as a means of identifying viruses, without the use of a virus-specific identification agent. In this work, infectious bronchitis virus (IBV) and three different influenza strains (Udorn, X31, PR8) were labelled with a mixture of green and red fluorescent DNAs using strontium chloride, and immobilised on glass slides for single molecule imaging using total internal reflection (TIRF) microscopy. The resulting fluorescent images were examined for colocalised fluorescence signal (spots displaying both green and red fluorescence), and these colocalised signals were subject to further analysis using a convolutional neural network algorithm.

Electron microscopy can detect the smallest of pathogens, but sample preparation is complex, and time-consuming, and only a tiny amount of material is sampled. This makes the technique unsuited to rapid diagnostic applications.

The COVID-19 pandemic has highlighted the unmet need for a pathogen detection method that is able to reliably, rapidly and simply detect a wide variety of pathogens in readily obtainable bodily fluid samples (such as saliva, nasal fluid, breath or urine), and has the potential to identify new pathogen types.

### SUMMARY OF THE INVENTION

In view of the foregoing, the present inventors have developed a general and rapid method for detecting and identifying intact pathogens from a sample of bodily fluid via fluorescence microscopy, using readily available non-specific reagents, with minimal sample preparation.

Specifically, in a first aspect, the invention provides a method of identifying pathogens in a sample using a fluorescence imaging system configured to illuminate the sample with an excitation light source and detect resulting fluorescence in multiple colour channels, comprising:
(1) a labelling step, involving incubating the sample with fluorescent probes from at least two of the following categories:
   (a) a fluorescent probe for binding to a pathogen surface carbohydrate;
   (b) a nucleic acid stain;
   (c) a membrane stain;
   wherein each category of fluorescent probe is detectable in a different one of said multiple colour channels of the fluorescence imaging system;
(2) an imaging step, involving imaging the sample using the fluorescence imaging system to produce fluorescence image data for each of the multiple colour channels;
(3) a measurement step, involving detecting the presence of candidate objects in the fluorescence image data which display fluorescence above a threshold, and recording the signal (preferably, signal intensity) in the multiple colour channels for each candidate object as sample data; and
(4) a characterisation step, involving analysing the sample data to generate result data, wherein the result data includes whether the candidate objects are pathogens and if so, the type of pathogen.

The technique is based on the fact that each pathogen type will vary in terms of the characteristics of its surface carbohydrates, nucleic acid material, and membrane material which will affect the overall fluorescence signal detected when the pathogens are labelled with fluorescent probes of types (a), (b) and (c). For example, different pathogen types will vary in terms of the types and amounts of carbohydrates at their surface, including the exact processing state of those carbohydrates (for example, the amount of underprocessed oligomannose at the pathogen surface, for the reasons described below) which will affect the amount of probe (a) which binds to the pathogen surface. Similarly, different pathogen types will vary in terms of the nature of their genetic material (RNA versus DNA, single stranded versus double stranded), the amount of the nucleic acid present, the accessibility of that nucleic acid material to fluorescent labelling (influenced by the nature of any encapsulating membrane(s) or capsid layer, the packing of the material, nucleoproteins etc), which will all contribute to differences in the amount of probe (b) that binds to those pathogens. Likewise, different pathogens will vary in terms of the extent of the membranes (generally correlating with the size of the pathogen) and the composition of their membranes (for example, in terms of the amount and distribution of cholesterol or gangliosides), which will affect the amount of probe (c) which binds to the membrane. As well as affecting the binding of the probes, the structural and compositional idiosyncrasies of a given pathogen can also contribute to differences in the fluorescence behaviour of probes (a)-(c), for example due to changes in the quantum yield (for example, probe (b) may have a higher quantum yield when bound to RNA instead of DNA, and probe (c) may display different quantum yields in different membrane environments) or Förster resonance energy transfer (FRET). Taking this multiplicity of factors into account, the fluorescence signal measured from a pathogen labelled with at least two of (a)-(c) [and most preferably all of (a)-(c)] serves as a highly distinctive “signature”, which the present invention exploits to identify the pathogen type. The technique is broadly applicable to pathogens, irrespective of type, and can even be used to identify and characterise previously unknown pathogens.

This approach should be contrasted with prior art techniques which identify pathogens based on fluorescent probes targeted to specific markers characteristic of a given pathogen, for example using antibodies or nucleic acid-specific probes. In such instances, the techniques identify pathogens based simply on whether a signal is present. However, this approach is uneconomical and impractical to scale into a broad diagnostic test for identifying different pathogens. In particular, the range of different probes which can be used is necessarily limited by the ability to distinguish between different fluorophores. In addition, such an approach is unsuitable for identifying new pathogen types, against which targeted probes have not yet been developed.

The method of the invention should also be contrasted with the technique taught in Shiaelis et al., which relies on the binding of non-specific fluorescent DNA probes to viral membranes via a cation-mediated mechanism. The Shiaelis technique probes only the viral membrane, and thus does not take advantage of the wealth of additional information that is available by combining information about the membrane with information about other parts of the pathogen, necessarily limiting the accuracy and scalability of the method. In addition, the nature of the interaction between DNA and membrane is poorly characterised, meaning that fluorescence characteristics cannot readily be linked back to actual compositional and physical features of the pathogens. Furthermore, the technique relies on single-stranded DNA (48 nucleotides for the “green” DNA and 72 nucleotides for the “red” DNA), purchased from a specialist supplier, which could potentially serve as a bottleneck in rapid scale-up of the technique (in much the same way as encountered for the primers required for conventional PCR detection methods).

Advantageously, the method of the present invention can obtain clinically meaningful results in a matter of minutes from readily obtainable bodily fluids, with minimal sample preparation, using cheap, non-specific reagents (for example, no specific antibodies or specific nucleic acid sequences). The process is also simple to run, and can be automated. Importantly, the method can be refined over time (as described in the learning section below), so that it is able to improve the detection of targeted pathogens, and adapts to new pathogens via software updates without any change to the instrumentation or reagents. These properties make the detection method compatible with pre-emptive mass testing and create a new capability to screen against unknown viruses without the need for any prior understanding of virus genetics or biology. If the method and the instrument supporting it are deployed ubiquitously, test results from all over the world can be reported to a centralised database so that countermeasures can be taken at the earliest signs of an outbreak.

Fluorescent Probes

The fluorescent probes in the present invention are generally chosen due to their ability to bind to a wide variety of pathogens.

For example, in the case of probe (a), pathogen surface proteins and lipids can display significant glycosylation. There is huge variation in surface glycoproteins and glycolipids between different pathogen types, but comparatively much less variation in the types of glycosylation motifs found on those proteins/lipids. Thus, targeting the glycosylation motifs through probe (a) means that the probe can attach to a broad spectrum of pathogen types, in contrast to antibodies developed against glycoproteins/glycolipids specific to a given pathogen type.

In the case of probe (b), different pathogens will generally have different amounts of nucleic acid material present. For example, virus genomes can range in size from 2 kb for Circoviridae to around 2 Mb for certain pandoraviruses. Furthermore, the accessibility of the nucleic acid material will vary between pathogen types—for example, the ability of a nucleic acid stain to reach nucleic acid material will be different for an enveloped virus containing a lipid bilayer compared to a non-enveloped virus lacking such a bilayer. By using a nucleic acid stain that is capable of staining nucleic acid material in a non-specific way (in other words, a general purpose nucleic acid stain) instead of a sequence specific stain it is thus possible for the probe (b) to be effective against any pathogen type.

In the case of probe (c), this again provides a general way of distinguishing pathogens based on membrane characteristics. For example, the probe (c) can be used to distinguish between enveloped viruses (having a membrane) and non-enveloped viruses (lacking a membrane). For pathogens which do include a membrane, the fluorescence intensity detected from probe (c) will generally depend on the composition of the membrane (due to the probe favouring specific environments [such as fluorescently-labelled cholesterol, like BODIPY-cholesterol, which targets cholesterol rich domains in membranes] or due to the composition affecting quantum yield and emission wavelength of the probe [such as Nile Red, which is bright orange in a non-polar lipid environment and far red around polar lipids]) and (in particular) the size of the pathogen, so that bigger pathogens will produce a brighter signal. In this regard, it should be remembered that many pathogens are typically comparable to or smaller than the diffraction limit of visible light, complicating determination of their size.

To better explain the ability to distinguish between different viruses, it is instructive to take coronavirus and influenza virus as illustrative examples. Both are enveloped viruses of approximately 100 nm—below the diffraction limit of light, and thus indistinguishable under normal white light illumination. As with most viruses, virus proteins on the exterior of coronavirus and influenza virus are glycosylated to hide the virus' peptide sequences from being recognized by the host immune system (the denser the “glycan shield” of the protein, the fewer peptide patches are available for immune recognition). Glycosylation is a multi-step process; usually oligomannose is attached first, with further processing resulting in cleavage of the mannose and attachment of other sugar groups such as galactose. Attaching more complex sugar groups results in the protein's glycosylation pattern being more like that of host proteins. However, a protein with a dense glycan shield will often have many “underprocessed” oligomannose groups remaining. This in turn can be a signature for the host immune system to recognize as pathogenic. Influenza hemagglutinin (HA) is an example of a surface protein that is dense in underprocessed glycans, whereas SARS-CoV-2 spike (S) protein has fewer glycans in total, but they are more complex sugars. Hence a fluorescent probe targeting oligomannose can be used to distinguish between influenza and SARS-CoV-2. Moreover, coronavirus has a genome size over double that of influenza virus (30 kb compared to 14 kb), providing more sites for incorporating nucleic acid stain, albeit that differences in accessibility of the genome to the nucleic acid stain may not lead to a direct correlation between genome size and labelling level with nucleic acid stain.

As another example, a non-enveloped virus such as adenovirus will exhibit further differences from either influenza or coronavirus. Most obviously, the lack of a viral envelope means membrane staining will be absent, however other features of non-enveloped virus biosynthesis will add further differences. Overall glycosylation is typically much lower in non-enveloped versus enveloped viruses due to the absence of transmembrane viral proteins that would progress through the primary glycosylation pathways of the ER and Golgi apparatus. The ratio of O- to N-linked glycosylation is also often higher, leading to substantial differences in the complexity of associated glycans. On top of this, the lack of an envelope to provide structural integrity means non-enveloped viruses typically have much more tightly associated protein capsids, which will reduce accessibility of nucleic acid dyes to viral generic material, thereby altering its staining profile. The nature of this material may also differ, as is the case with adenovirus (dsDNA genome) versus influenza (ssRNA genome), for example. All of these differences will result in characteristics differences in the fluorescence “signature” when the viruses are labelled with probes (a)-(c).

The labelling step may use any combination of the fluorescent probes, for example (a) and (b), (a) and (c), (b) and (c). In preferred implementations, the labelling step uses (a) and (b)/(c).

Advantageously, carbohydrate labelling provides a particularly useful means of distinguishing between different pathogens due to its heavy dependence on surface chemistry, unlike membrane staining and nucleic acid staining which are more (although not exclusively) a function of the size of the pathogen and its genome.

Most preferably, the labelling step involves incubating the sample with fluorescent probes from all of categories (a), (b) and (c).

It is possible for multiple fluorescent probes of a given category to be present. In particular, it can be useful for there to be multiple (for example, 2 or 3) fluorescent probes in category (a) which bind to different carbohydrates to increase the breadth and accuracy of pathogen identification. Each probe in category (a) is detectable in a different one of said multiple colour channels. Generally, there will only be one fluorescent probe in category (b) or (c), since the binding of these types of fluorescent probe is typically more general, and thus there is generally less information derivable from using multiple probes in these categories.

In a preferred implementation, there may be two (or more) different fluorescent probes in category (a), targeting different pathogen surface carbohydrates, one fluorescent probe in category (b), and one fluorescent probe in category (c), each probe detectable in a different one of said multiple colour channels of the fluorescence imaging system. Advantageously, probing four different components of pathogens in this way allows the technique to distinguish between a large range of different pathogen types, with high accuracy and sensitivity.

Suitably, the method is carried out using fluorescent probes having a known number of fluorophores per probe. Preferably, there is one fluorophore per fluorescent probe. In instances where the fluorescent probe contains more than one fluorophore, the fluorescent labelling level of the fluorescent probe is preferably characterised. In particular, the labelling level may be characterised by the average number of fluorophores per fluorescent probe (for example, the mean number) and/or the distribution of labelling levels for the fluorescent probe. In instances where the fluorescent probe (a) is a protein labelled with fluorophores, the labelling level may be determined using absorption spectroscopy according to methods known to the person skilled in the art (see, for example, the note “Calculate dye:protein molar ratios” available on the ThermoScientific website at http://tools.thermofisher.com/content/sfs/brochures/TR0031-Calc-FP-ratios.pdf).

Fluorescent Probe (a)

Fluorescent probe (a) is a fluorescent probe for binding to a pathogen surface carbohydrate.

The probe (a) may be, for example, a mannose-binding probe, a fucose-binding probe, a galactose-binding probe, an N-acetylglucosamine-binding probe, N-acetylgalactosamine-binding probe, a sialic acid binding probe, or a glucose-specific probe. By “mannose-binding probe” we mean a probe with specificity for mannose, or motif incorporating mannose, with analogous definitions applying in respect of the other probe types set out in the preceding sentence.

Preferred classes of probe (a) include mannose-binding probes, fucose-binding probes, sialic acid-binding probes. Mannose-binding probes are particular useful, since (as mentioned above) glycosylation processes usually start with the attachment of oligomannose, and it is typical for at least a portion of these oligomannose to remain unmodified (or “underprocessed”) dependent on the pathogen type. Thus, mannose-binding probes will be capable of binding to a broad spectrum of pathogen types, whilst also being sensitive to pathogen type due to the level of underprocessed oligomannose remaining at the surface.

The probe (a) may be a protein.

Preferably, the or each fluorescent probe (a) is a fluorescently labelled lectin. As the skilled reader will be aware, lectins are proteins that bind to specific carbohydrate structures, but lack enzymatic activity. Advantageously, lectins can have broad binding capability to different pathogens, and are generally readily available from natural sources. In implementations incorporating two or more fluorescent probes (a) it is preferred that each probe (a) is a fluorescently labelled lectin.

The lectin may be, for example, a mannose-binding lectin, a fucose-binding lectin, a galactose-binding lectin, an N-acetylglucosamine-binding lectin, N-acetylgalactosamine-binding lectin, a sialic acid binding lectin, or a glucose-specific lectin. By “mannose-binding lectin” we mean a lectin with specificity for mannose, or motif incorporating mannose, with analogous definitions applying in respect of the other lectin types set out in the preceding sentence. Examples of suitable lectins are provided in Table 1 below.

TABLE 1
Lectins suitable for use as probe (a)

| Name | Specificity |
|---|---|
| Agaricus bisporus Lectin (ABA/ABL) | Galactoseβ1-3N-Acetylgalactosamine |
| Aleuria aurantia Lectin (AAL) | Fucose |
| Allium sativum Lectin (ASA) | Mannose |
| Amaranthus caudatus Lectin (ACL/ACA) | Galβ3GalNAc |
| Artocarpus integrifolia Lectin (AIA) | Galβ3GalNAc |
| Bauhinia purpurea Lectin (BPL/BPA) | Galβ3GalNAc |
| Concanavalin (ConA) | α- mannose, α-glucose |
| Crotalaria juncea Lectin (CJL) | Gal > GalNAc |
| Datura stramonium Lectin (DSA/DSL) | (N-Acetylglucosamine)2-4 |
| Galanthus nivalis Lectin (GNL/GNA) | α-Mannose |
| Hippeastrum hybrid Lectin (HHL) | α-Mannose |
| Iris hybrid Lectin (IRA) | N-Acetylgalactosamine |
| Lycopersicon esculentum Lectin (LEL/LEA) | (N-Acetylglucosamine)2-4 |
| Maackia amurensis Lectin (MAA/MAL I) | Siaα2-3Galβ1-4GlcNAc |
| Maackia amurensis Lectin (MAA/MAL I + II) | α2-3 Linked Sialic Acids |
| Maackia amurensis Lectin (MAA/MAL II) | Siaα2-3Galβ1-3GalNAc |
| Maclura pomifera Lectin (MPL/MPA) | Galβ3GalNAc |
| Morus rubra Lectin (MRL) | Galactose, N-Acetylgalactosamine |
| Narcissus pseudonarcissus Lectin (NPL/NPA) | α-Mannose |
| Psophocarpus tetragonolobus Lectin (PTL/PTA I) | N-Acetylgalactosamine, Galactose |
| Sambucus nigra Lectin (SNA/EBL I) | α2-6 Sialic Acid |
| Sambucus nigra Lectin (SNA/EBL I + II) | Sialic Acid |
| Sambucus nigra Lectin (SNA/EBL II) | N-Acetylgalactosamine > Galactose |
| Solanum tuberosum Lectin (STA/STL) | (N-Acetylglucosamine)2-4 |
| Tulip sp. Lectin (TL) | Complex N-Glycans, Mannose |
| Vicia ervilia Lectin (VEA) | Mannose, Glucose |
| Vicia faba Lectin (VFA) | Mannose |
| Vigna radiate lectin (VRA) | Galactose |
| Wheat Germ Agglutinin (WGA) | N-Acetylgalactosamine, sialic acid |
| Wisteria floribunda Lectin (WFA/WFL) | N-Acetylgalactosamine |

The above lectins are available from companies such as Glycomatrix (Dublin, Ohio, USA).

Preferably, the lectin does not rely on the presence of divalent ions for binding. In particular, this is because the level of divalent ions can vary between bodily fluid types and even between samples of bodily fluid of the same type (in particular saliva), which can perturb binding. Furthermore, the pH of bodily fluids such as saliva is buffered by phosphates and bicarbonate, and the presence of these components alongside divalent ions can cause complex aggregation behaviour (the combination of phosphate and divalent ions generally driving aggregation, which is somewhat counteracted by the presence of bicarbonate).

Preferred classes of lectin include mannose-binding lectins, fucose-binding lectins, sialic acid binding lectins. Mannose-binding lectins are particular useful, since (as mentioned above) glycosylation processes usually start with the attachment of oligomannose, and it is typical for at least a portion of these oligomannose to remain unmodified (or “underprocessed”) dependent on the pathogen type. Thus, mannose-binding lectins will be capable of binding to a broad spectrum of pathogen types, whilst also being sensitive to pathogen type due to the level of underprocessed oligomannose remaining at the surface.

Particularly preferred examples of lectins for use in the invention include GNA, AAL, MAA since these lectins bind to motifs found across a broad spectrum of pathogens. Most preferably, the lectin is or includes GNA, since GNA binds to mannose (with the resulting benefits mentioned above) and does not rely on the presence of divalent ions for binding.

As an alternative, the fluorescent probe (a) may be or include a fluorescently labelled antibody, antibody fragment (such as a Fab), or aptamer with specificity for a carbohydrate epitope commonly present on glycoproteins and glycolipids at a pathogen's surface. However, preferably the fluorescent probe (a) is not an antibody, antibody fragment, or aptamer due to the expense and complication of obtaining such reagents.

In addition, the fluorescence probe (a) may be or include fluorescently labelled cholera toxin subunit B (CTXB). CTXB binds to sialic residues on GM1 gangliosides.

Any suitable fluorophore may be used to label the fluorescent probe (a), provided that its emission can be distinguished from the emission of the other fluorescent probes present in the sample. Suitable fluorophores include, for example, dye molecules, such as Alexa Flour® dyes available from Thermo Fisher Scientific Corporation, Atto dyes available from Atto-Tec, or cyanine dyes such as Cy3, Cy5 or Cy7 also available from Thermo Fisher Scientific Corporation.

Fluorescent Probe (b)

Fluorescent probe (b) is a fluorescent nucleic acid stain. The skilled reader is aware of a range of suitable nucleic acid stains, commercially available from providers such as Thermo Fisher Scientific Corporation.

In assays looking for the presence of human or animal viruses, the nucleic acid stain is preferably capable of staining RNA. In assays looking for the presence of bacteria, the nucleic acid stain is preferably capable of staining DNA. Most preferably, the nucleic acid stain is capable of staining both DNA and RNA.

Suitably, the nucleic acid stain is a membrane-permeable dye, to facilitate its use with intact bacteria and enveloped viruses.

The nucleic acid stain may be, for example a cyanine dye capable of binding both DNA and RNA. Examples of suitable membrane-permeable cyanine dyes include, for example, the SYTO dyes sold by Thermo Fisher Scientific Corporation and, including blue fluorescent stains (such as SYTO 40, SYTO 41, SYTO 42, SYTO 45), green fluorescent stain such as (SYTO 9, SYTO 10, SYTO 11, SYTO 12, SYTO BC, SYTO 13, SYTO 14, SYTO 16, SYTO 21, SYTO 24, SYTO 25, or SYBR #green I), orange fluorescent SYTO dye (such as SYTO 80-85), and red fluorescent SYTO dye (such as SYTEO 17 or any of SYTO 59-SYTO 64). Other alternatives include SYBR stains, for example SYBR Green I/II (showing stronger staining of DNA than RNA).

Stains with greater specificity for DNA over RNA include, for example, Vybrant DyeCycle stain (such as Vybrant DyeCycle Violet Stain and Vybrant DyeCycle Green Stain), bis-benzimide dyes (a Hoechst stain such as Hoechst 33258, Hoechst 33342 or Hoechst 3458), SYBR Safe, Acridine Orange, and 4′6-diamidino-2-phenylindole (DAPI).

Suitable stains with greater specificity for RNA over DNA include, for example, SYTO RNASelect green fluorescent stain, or SYBR Green II RNA (although the latter still shows some binding to DNA as well).

Preferably, the nucleic acid stain is a dye which displays fluorescence enhancement upon binding to a nucleic acid (in other words, a dye whose quantum yield is relatively low when in its free form but increases significantly upon binding to a nucleic acid). For example, the nucleic acid stain may have a quantum yield of 0.1 or less, preferably 0.05 or less, more preferably 0.01 or less when in its free (unbound form) and a quantum yield of 0.3 or more, preferably 0.4 or more, more preferably 0.5 or more in its bound form. Advantageously, stains having this characteristic reduce fluorescent background during imaging, and therefore improve the signal:noise ratio of detected pathogens.

Fluorescent Probe (c)

Fluorescent probe (c) is a fluorescent membrane stain. The membrane stain may be a lipophilic molecule which inserts into the membrane of a pathogen.

The membrane stain may be a cyanine dye incorporating lipophilic moieties, for example, a long-chain dialkyl carbocyanine dye or dialkylamino styryl dye. Suitable long-chain dialkylcarbocyanines include Dil, DilC₁₂, DilC₁₆, DiO, DiOC₁₆, DiD, and DiR, and suitable dialkylaminostyryl dyes include DiA and 4-Di-10-ASP, all available from Thermo Fisher Scientific Corporation. Other suitable membrane stains include, for example, PKH67, sold by Sigma Aldrich, Nile Red, or BODIPY-cholesterol.

Preferably, the membrane stain is a dye which displays fluorescence enhancement upon binding to a membrane (in other words, a dye whose quantum yield is relatively low when in its free form, but increases significantly upon binding to a membrane). For example, the membrane stain may have a quantum yield of 0.1 or less, preferably 0.05 or less, more preferably 0.01 or less when in its free (unbound form) and a quantum yield of 0.3 or more, preferably 0.4 or more, more preferably 0.5 or more in its bound form. Advantageously, stains having this characteristic reduce fluorescent background during imaging, and therefore improve the signal to noise ratio of candidate objects.

Preferably, the membrane stain is an environment-sensitive dye, which target parts of a membrane having certain characteristics (for example, specific molecules in the membrane, or areas having certain compositions) and/or displays differences in fluorescence intensity dependent on the local membrane environment. Examples of such dyes are discussed in Demchenko et al. “Monitoring Biophysical Properties of Lipid Membranes by Environment-Sensitive Fluorescent Probes”, Biophys. J. 96(9): 3461-3470 (2009). As an example, the membrane stain may be or include a membrane stain such as a fluorescently labelled cholesterol, such as BODIPY-cholesterol, which associates with cholesterol-rich membranes.

Suitably, the sample contains no, or only very low levels, of micelles/liposomes of the membrane stain during the imaging step. Whilst such particles can generally be discriminated from pathogens due to the lack of fluorescence from the one or more other categories of stain, it is best to avoid their presence altogether to simplify the imaging and data analysis. Preferably, the presence of micelles is minimised/avoided by using a membrane stain that does not spontaneously form micelles under the conditions during the incubation step and, in particular, avoiding the use of a membrane stain that requires formation of micelles/liposomes of the membrane stain to facilitate its addition to the pathogen membrane.

For membrane stains capable of forming micelles, the avoidance of micelles can be achieved by including the stain below the critical micelle concentration (CMC) and/or including a low level of surfactant to disrupt micelle formation (for example, a low level of Triton-X, e.g. 0.1 vol % or less, or 0.05 vol % or less). Since spontaneous micelle formation by zwitterionic lipophilic stains is driven by the hydrophobic effect, interventions that reduce the potency of this effect will tend to increase the CMC of such stains and thereby reduce the frequency and stability of such micelles in the sample. This can be achieved in a number of ways, including increasing the ionic strength of the solution (e.g. by addition of a salt, such as NaCl or KCl), adding chaotropic agents (e.g. urea), or reducing pH below 7.0.

Combinations of Fluorescent Probes

In the present invention, the at least two fluorescent probes from categories (a)-(c) must be distinguishable by the fluorescence imaging system. Thus, a specific combination of compatible probes must be chosen which can be properly imaged.

This requirement is evident from the definition above for the fluorescent probes to be detectable in a different one of the multiple colour channels of the fluorescence imaging system. This requirement may be achieved by choosing fluorescent probes whose emission spectra are positioned at different wavelength bands. To aid resolution of the different fluorophores, the emission spectra of the different fluorophores preferably have regions of relatively low overlap. Differently, and simply, stated—the fluorophores of the fluorescent probes have different emission colours (i.e. produce different colours of fluorescent emission), such that their fluorescence emission can be separated using optical filters (dichroic filters, longpass filters, bandpass filters).

For example, the first probe may be a green probe and the second probe may be an orange or red probe (the colours referring to the predominant colour of fluorescence emission). In systems using three fluorescent probes, the first probe may be a green probe, the second probe may be an orange probe, and the third probe a red probe. In systems adding a fourth fluorescent probe, this may be in the far red or near-infrared wavelength region. In practice, the fluorescent emission of different probes can overlap to some extent, but the skilled reader is familiar with how to address and (if appropriate) correct for this issue.

Examples of compatible combinations include the following:

| Combi- | Fluorescent | Fluorescent | Fluorescent |
|---|---|---|---|
| nation | probe (a) | probe (b) | probe (c) |
| 1 | GNA labelled with Cy5 | Syto 13 | Dil |
| | (excited at 640 nm) | (excited at | (excited at |
| | And/or | 488 nm) | 561 nm) |
| | AAL labelled with Cy7 | | |
| | (excited at 750 nm) | | |
| 2 | GNA labelled with | Syto 61 | Nile Red |
| | Alexa 488 | (excited at | (excited at |
| | (excited at 488 nm) | 640 nm) | 561 nm) |

Pathogen Type

Suitably, the method of the invention is used for identifying bacteria or virus in a sample.

Suitably, the “type of pathogen” identified in the characterisation step may be a pathogen family, a pathogen sub-family, a pathogen genus, or a pathogen species.

In the case of bacteria, the “type of bacteria” may be a bacterial phylum, a bacterial class, a bacterial order, a bacterial family, a bacterial sub-family, a bacterial genus, a bacterial species, or a bacterial strain.

Preferably, the method is used for identifying a virus. In such instances, “type of virus” in the characterisation step may involve determining the virus family, preferably the virus subfamily, more preferably the virus genus, most preferably the virus species (these terms being used in accordance with the five-rank classification assigned structure of the International Classification on Taxonomy of Viruses). The type of virus may also be the viral strain. The “type of virus” may also be identification of whether the virus is an enveloped virus or non-enveloped virus, based on the level of signal produced from fluorescent probe (c).

Preferably, the method is used to distinguish between viruses selected from at least two of (preferably three of, preferably all of) coronavirus, respiratory syncytial virus (RSV), influenza, rhinovirus, and adenovirus. In such instances, the “pathogen type” assigned in characterisation step is or includes a label selected from coronavirus, respiratory syncytial virus (RSV), influenza, rhinovirus, and adenovirus.

Preferably, the method is used to identify whether a sample contains a coronavirus, more preferably to assess whether a sample contains SARS-CoV-2. In such instances, the “pathogen type” assigned in the characterisation step may be “SARS-CoV-2” or “not SARS-CoV-2/unclassified”.

Preferably, the method is used to identify (and distinguish) coronavirus types, for example, SARS-CoV-1, MER-CoV, SARS-CoV-2, HCoV-NL63, HCoV-229E, HCoV-OC43 and HCoV-HKU1. In such instances, the “pathogen type” assigned in characterisation step is or includes a label selected from SARS-CoV-1, MER-CoV, SARS-CoV-2, HCoV-NL63, HCoV-229E, HCoV-OC43 and HCoV-HKU1, or none of the above/unclassified.

In addition to characterising the type of pathogen, the method may optionally be used to characterise the infection profile of the pathogen. By “infection profile”, we mean details about the nature of the infection of the source (patient) from which the pathogen was obtained, such as the origin of the pathogen from within the patient (the cell type or tissue type in which the pathogen was located) and the time course of the infection (day 1, day 2, day 3, and so on). Regarding the origin of the pathogen, a given pathogen can typically infect a range of different cells or tissues, which can result in subtle but detectable differences in staining behaviour dependent on the cell/tissue. In particular, glycosylation profiles of a pathogen can vary dependent on the tissue in which the pathogen was located, due to the differences in the underlying regulation of glycosylation in those tissues. In the case of viruses, the membranes of enveloped viruses are typically derived from portions of the host cell membranes, and thus can change dependent on the composition of the host cell. Regarding the time course of infection, the nature of the pathogen can change over the course of an infection, both due to development of the pathogen itself, but also due to changes in the interaction between host and pathogen—in particular, the effect of the immune system as it encounters and begins to target the pathogen. This again can lead to changes in staining behaviour, for example, with antibodies sterically blocking access of the carbohydrate probe to carbohydrates on the pathogen surface. It should be noted that the compositional and structural differences arising from different infection profiles of a given pathogen type will generally be much more subtle than compositional and structural differences between pathogen types, and are beyond the capabilities of conventional diagnostic assays. However, the high accuracy and sensitivity available using methods of the present invention allow this additional information to be uncovered, particularly in the case of embodiments incorporating all of probes (a), (b) and (c) [even more so when two or more probes of category (a) are employed].

Sample Type

Preferably, the sample is a bodily fluid obtained from a human or animal, preferably a human.

The bodily fluid may be, for example, saliva, nasal fluid, sweat, breath, urine, semen, cervical mucus, or blood. Most preferably the bodily fluid is saliva or nasal fluid, since these fluids can provide high levels of pathogen, whilst also having relatively low levels of human cells that might complicate sample preparation and analysis.

Sample Preparation

The present invention involves the detection of intact pathogens. In other words, the pathogens are not subjected to lysis during the sample preparation step.

The bodily fluid may be purified, for example, by filtering or centrifugation. Preferably, this purification occurs before labelling, to avoid unwanted components of the sample scavenging the fluorescent probes.

Preferably, the sample is filtered prior to the incubation step. Such filtration preferably removes non-pathogenic cells (endogenous cells), which can otherwise become labelled with probes (a)-(c) and complicate identification of pathogens. Additionally or alternatively, such filtration may remove pathogen aggregates, which can otherwise complicate analysis. Generally, the filtration is carried out with a filter having an average pore size between about 0.2 μm to about 2 μm, more preferably 0.3 μm to 1 μm, more preferably 0.3 to 0.8 μm, most preferably 0.4 μm to 0.5 μm. The filter may be, for example, a nylon membrane filter, such as a Fisherbrand™ Nylon Membrane Filter available from Thermo Fisher Scientific.

Preferably, the bodily fluid is diluted with a diluent before imaging to reduce the effects of variation in pH and concentration of complicating components (such as divalent ions, which can cause aggregation of viruses) on the obtained results. Dilution can also serve to minimise the effects of differences in viscosity between samples (which can arise from variation in the viscosity of different bodily fluid types, as well as viscosity differences between patients), which simplifies calculation of diffusion characteristics such as the hydrodynamic radius (discussed below). Furthermore, dilution of the sample can also serve to decrease background fluorescence, and ensure that the concentration of pathogen is at a suitable level—that is sufficiently low to allow individual pathogens to be resolved but sufficiently high to allow data to be collected at a reasonable rate.

The level of dilution, as expressed in terms of volume of bodily fluid:volume of diluent, may be, at least 1:3, at least 1:5, at least 1:7, at least 1:10, or at least 1:20. Preferably, the level of dilution is between 1:5 and 1:10.

The diluent may be any suitable buffer, such as HEPES buffered saline (HBS).

Suitably, the buffer is an imaging buffer, having an oxygen-scavenging system to improve the fluorescence signal from probes (a)-(c), for example by reducing the rate of photobleaching. Suitable oxygen-scavenging systems include, for example, the “GLOX” system comprising catalase, glucose and glucose oxidase in combination with a reducing and oxidizing agent (for example, Trolox and its quinone derivative).

Optionally, the dilution step occurs before filtration, to simplify the filtration process.

Optionally, the diluent is free of probes (a)-(c) [in others words, dilution occurs separately from and before incubation with probes (a)-(c)]. Alternatively, the diluent contains probes (a)-(c). In instances where the diluent contains probes, the filtration step is preferably carried out before dilution, for the reasons set out above.

Optionally, a reagent is added to lyse extracellular vesicles (EVs), without causing lysis of the pathogens. For example, a low-level of non-ionic surfactant (for example, up to 0.05% or 0.1% by volume of the sample), such as Triton-X, may be added to achieve this. Alternatively, the sample is used without lysing EVs, since EVs can themselves provide a useful signature of the particular pathogen present (for example, virus-infected cells can incorporate viral surface proteins on EVs) and may become labelled with fluorescent probes (a)-(c), which can be factored into the determination of the pathogen type. For example, the sample preparation step may not involve the addition of any surfactants (in other words, the diluent may be a surfactant-free diluent).

Optionally, additional components are added to inactivate or disrupt non-pathogenic components of the bodily fluid which might otherwise complicate analysis. For example, in the case of saliva, additional components may be added to disrupt the mucin network, which can otherwise bind to virus and interfere with the assay. To this end, the saliva sample may be treated with a redox reagent (such dithiothreitol) to disrupt disulphide bonds between mucins, and/or treated with a chelator such as EDTA to remove calcium ions which mediate links between mucins. However, preferably the sample is not treated with such additional components (in particular, without a chelator) to simplify sample preparation and avoid a potential source of variation between assay runs. In this regard, dilution is generally sufficient to overcome difficulties created from non-pathogenic components in the bodily fluid.

In instances where fluorescent probe (a) is a lectin whose binding is mediated by divalent ions, the diluent may include the relevant divalent ions (for example, Ca²⁺, Zn²⁺ or Mn²⁺). However, as noted in the discussion of fluorescent probe (a) above, it is preferred to use a lectin whose binding is not mediated by divalent ions, and thus the diluent may be substantially free of divalent ions. Advantageously, this can limit the amount of pathogen aggregation caused by divalent ions present in the sample.

Preferably, the sample preparation step does not include the addition of any further components beyond a buffer (optionally including an oxygen-scavenging system) and relevant probes (a)-(c). Advantageously, this limits the number of reagents required to run the assay, thus simplifying sample preparation, reducing costs and helping to minimise potential sources of variation that could affect consistency of results.

Optionally, the sample preparation step consists of a filtration step followed by dilution with a diluent consisting of a buffer and the two or more fluorescent probes (a)-(c).

Optionally, the sample preparation step consists of a dilution step followed by a filtration step, followed by addition of the two or more fluorescent probes (a)-(c). This method is particularly suited to saliva samples, which are relatively viscous and benefit from dilution before filtering.

Preferably, the labelling step is relatively short in duration, to minimise the overall run time of the assay. The labelling step may comprise, for example, no more than 10 minutes from addition of the probes to initiating the imaging step, preferably no more than 8 minutes, more preferably no more than 7 minutes, more preferably no more than 6 minutes, more preferably no more than 5 minutes, more preferably no more than 3 minutes.

Imaging Step

In the imaging step the sample is illuminated with excitation light sources so as to cause excitation of the fluorescent probes, with subsequent detection of the resulting fluorescence emission.

Fluorescence imaging systems suitable for carrying out the invention may comprise:
an excitation light source, preferably operable at a range of wavelengths, suitable for exciting the fluorescent probes;
imaging optics (e.g. an objective lens); and
a detection system (including detection optics, e.g. a camera for detecting fluorescence, and preferably optical filters to remove unwanted scatter and background from fluorescence signal).

Suitably, the imaging is wide-field imaging. To this end, the imaging step involves capturing fluorescence images of candidate objects in the sample using a camera, for example a charge-coupled device (CCD), such as an electron-multiplying CCD (EMCCD), or a complementary metal-oxide semiconductor (CMOS) camera. Advantageously, wide-field imaging allows signals from multiple candidate objects to be viewed simultaneously, increasing acquisition rate. Forming an image of each candidate object also allows much more information about the candidate object to be obtained—allowing measurement of the size of the object (for example, through characterisation of the point spread function of the object), diffusion characteristics of individual objects, and facilitating resolution of closely spaced objects (resolving two close objects as separately signals, instead of a single combined signal) in a way not possible with point source detectors, such as those used for flow virometry.

The dilution level of the sample is such as to allow single candidate objects to be resolved (whether that be single pathogens or aggregates of pathogens), with a low probability of multiple candidate objects overlapping in a given image.

Preferably, the magnification level of the objective lens, frame rate and exposure time of the camera, and the illumination period (duty cycle) if pulsed illumination is used, are chosen so as to focus the emission associated with a single candidate object in a given frame and colour channel onto a relatively small number of pixels, for example with 90% of the fluorescence (overall photon counts after subtraction of background) for a given candidate object falling on an area of 49 pixels or fewer (7×7), 36 pixels or fewer (6×6), 25 pixels or fewer (5×5), 16 pixels or fewer (4×4), 9 pixels or fewer (3×3), or 4 pixels (2×2). Focussing the emission onto a relatively small number of pixels has a number of associated advantages. For example, it increases the signal to noise of detected fluorescence by minimising the influence of camera read-noise and background shot noise. This can allow relatively low excitation power densities to be used (which decreases the rate of photobleaching) and/or allow the material to be flowed at a faster rate whilst still generating suitable signal levels (as discussed below). Moreover, it simplifies calculation of the signal intensity associated with a candidate object. Finally, focussing the emission from a candidate object onto a relatively small number of pixels allows a high density of candidate objects to be viewable simultaneously, without signals overlapping.

Differently stated, the majority of the point spread function at the focal plane of the fluorescence imaging system may be calculated to occupy a relatively small number of pixels. For example, 90% of the point spread function of the fluorescence imaging system may fall within 49 pixels or fewer (7×7), 36 pixels or fewer (6×6), 25 pixels or fewer (5×5), 16 pixels or fewer (4×4), 9 pixels or fewer (3×3), or 4 pixels (2×2).

Suitably, the objective lens is an air objective lens, to simplify construction of the fluorescence imaging system. The objective lens may, for example, provide 10× or more magnification, 20× or more magnification, or 40× or more magnification. The numerical aperture (NA) of the objective lens may be, for example, 0.2 or greater, 0.3 or greater, 0.4 or greater, 0.5 or greater, 0.6 or greater, 0.7 or greater, or 0.8 or greater. A preferred implementation involves the use of an air objective having a 20× magnification and NA of 0.45, since that provides a suitable sized field of view, photon detection rate and depth of focus. Such air objectives also have the advantage of being relatively cheaper and simpler to implement and maintain than oil-based objectives.

The frame rate of the camera may be, for example, at least 30 frames per second (fps), at least 40 fps, at least 50 fps, at least 60 fps, at least 70 fps, at least 80 fps, at least 90 fps, or at least 100 fps. Preferably, faster frame rates facilitate collection of the emission associated with a single candidate object onto a small area, providing the advantages detailed above.

In instances in which the candidate objects are so heavily labelled that the camera becomes saturated, the frame rate may be increased and/or exposure time decreased to avoid saturation, although in such instances an adjustment may need to be made to compare against data taken with a different frame rate/exposure time (assuming that the photon count of a given pixel doubles as the exposure time doubles). Additionally or alternatively, the illumination period may be decreased if pulsed illumination is used.

The excitation light source preferably comprises one or more lasers chosen to match the excitation spectra of the fluorescent probes. In certain instances, one laser may be capable of exciting multiple (potentially all) of the different fluorescent probes (a)-(c). More normally, however, the excitation light source comprises multiple lasers which each excite one, or only a subset, of the different fluorescent probes (a)-(c). For example, the excitation light source may include any combination of a first laser operating below 500 nm (for example, 350 nm-500 nm), a second laser operating between 500-600 nm, a third laser operating between 600-700 nm, and a fourth laser operating above 700 nm. For example, the excitation light source may include lasers operating at 488 nm, 561 nm, 640 nm and/or 750 nm. Preferably, the fluorescence imaging system incorporates lasers capable of emission at three or more wavelengths, optionally four or more wavelengths.

Illumination of the sample may be carried out using any suitable wide-field illumination mode, for example, widefield epifluorescence microscopy (in which the excitation light passes through the objective lens) or light sheet fluorescence microscopy (in which the excitation light source produces a sheet of light illuminated laterally at and parallel to the focal plane of the objective lens). Preferably, the excitation light source produces a sheet of light illuminated laterally at and parallel to the focal plane of the objective lens, since this minimises the contribution of fluorescence from out of focus objects (in particular, unbound fluorescent probes and candidate objects), helping to improve signal to noise.

In such instances, the thickness of the light sheet (measured parallel to the central axis of the objective lens) as it intercepts the focal volume is preferably comparable to the thickness of the focal volume of the objective lens. For example, the thickness of the light sheet may be less than or equal to the focal volume. In practice, the thickness of the light sheet at the point at which intercepts the central axis of the objective lens may be, for example, 20 μm or less, 15 μm or less, 10 μm or less, or 5 μm or less (as measured parallel to the central axis of the objective lens).

When the excitation light source operates at multiple excitation wavelengths, the light sheets for different excitation wavelength may have a high degree of overlap at the point where they intersect the central lens axis (for example, greater than 80% cross-sectional overlap, or greater than 90% cross-sectional overlap, as viewed along the light sheet). For example, the different excitation wavelengths may overlap in at least 70% of the focal volume, at least 80% of the focal volume, or at least 90% of the focal volume.

Alternatively, the light sheets for different excitation wavelengths may be stacked on top of one another, with relatively low or no overlap at the point where the sheets intersect the central lens axis. The stacked arrangement is particularly advantageous in instances where the chromatic aberration of the objective lens is such that the focal volumes associated with the different emission wavelengths are axially shifted relative to one another. In such instances, the light sheets are preferably stacked to reflect the chromatic aberration characteristics of the objective lens. This implementation is particularly advantageous when flowing sample vertically through the sample volume (either directly parallel, or at an acute angle, as discussed below), since the stacked arrangement allows sequential excitation at different wavelengths without the use of pulsed illumination.

The combined thickness of the volume illuminated by the light sheets at multiple excitation wavelengths may be, for example, 40 μm or less, 30 μm or less, 20 μm or less, 15 μm or less, μm or less, or 5 μm or less.

The imaging step requires detecting the fluorescence of each candidate object across multiple colour channels.

Optionally, the imaging step involves detecting fluorescence in the individual colour channels simultaneously.

This may be achieved by detecting fluorescence emission in different colour channels on separate pre-determined detector areas.

In one implementation, this is achieved by splitting the emission in different colour channels to separate cameras. However, whilst this retains a large field of view, it results in a relatively bulky and expensive construction, particularly if scaling to 3 or 4 different colour channels.

Alternatively, multiple colour detection may be achieved on a single camera by configuring distinct portions of the detector to detect different colour channels. For example, for two-colour channel imaging the emission may be split so that one colour channel is directed to one half of the camera detector, and another camera channel is directed to the other half of the camera detector. For, three or four colour imaging, the camera detector may be split into quarters, in an analogous fashion. The skilled reader is aware of how to achieve this using suitable optical components, and commercially available splitters are available to achieve this configuration, such as the Dual-View™ and Quad-View™ systems from Optical Insights, LLC. Advantageously, this approach allows simultaneous imaging across multiple colours, and thereby can potentially permit additional information to be obtained by Förster resonance energy transfer (FRET) imaging. In addition, it can permit ratiometric imaging, in which emission from a given fluorescent probe is detectable across multiple colour channels, with a characteristic ratio of fluorescence in the multiple colour channels allowing the fluorescent probe to be identified. To give an example of ratiometric imaging, consider probes A and B, wherein excitation at a wavelength of 488 nm causes probe A to produce fluorescence in colour channel X, and probe B to produce fluorescence in colour channels X and Y, according to an intensity ratio Z. With knowledge of the intensity detected in channel Y and ratio Z, the individual contributions of probes A and B to the signal in channel X can be calculated. This can allow the use of more probes, without increasing overall complexity of the imaging system.

Additionally, or alternatively, imaging in multiple colour channels can be achieved or aided through use of a dispersive element (such as a prism or grating) to separate light signals into different wavelengths such that different wavelengths illuminate different parts of a detector.

In preferred implementations the dispersive element is a prism. The prism is preferably a compound prism, such as a doublet compound prism. A doublet compound prism may take the form of two wedge prisms fused/cemented along a shared facet such that their apex angles face away from one another. Advantageously, prisms can provide a compact structure for achieving dispersion with a combination of lower photon loss and lower (or no) deviation of emission compared to gratings.

In implementations incorporating a dispersive element (in particular a prism), the point spread function (PSF) may be asymmetric due to the asymmetric spectra of emission from different fluorophores. Different fluorescent labels will have different shapes for the PSF. Optionally, the shape of the PSF is used to detect and distinguish multiple fluorescent labels. This can enable the detection and distinction of all fluorescent labels of different colours simultaneously.

For example, consider a situation where the target biochemical component is labelled with one of a first fluorescent label or a second fluorescent label, the fluorescence emission from which is detectable on the same colour channel of a detector. In the absence of a dispersive element, the fluorescence from the two fluorescent labels may be indistinguishable due to them having the same PSF in the colour channel of the detector. However, with the dispersive element (prism) present, the PSF of the two fluorescent labels is different, allowing the fluorescent labels to be distinguished.

Further details about the use of a dispersive element to distinguish between different colour fluorophores can be found in the applicant's earlier application PCT/EP2021/064400. In particular, FIG. 6 of PCT/EP2021/064400 provides an explanation of the operation of the doublet compound prism, and FIGS. 7A-7C give an explanation of the resolution and identification of individual spectra for two different dyes simultaneously.

Another alternative for achieving simultaneous imaging in multiple colour channels can be achieved through the combination of lateral flow and pulsed excitation sources in the manner described below.

Alternatively or additionally, the imaging step involves detecting fluorescence in at least a subset (optionally all) of the multiple colour channels sequentially.

This may be achieved by using an excitation light source which switches between different wavelengths, and detecting the emission associated with each excitation wavelength sequentially. For example, the fluorescence imaging system may include two or more pulsed lasers operating at different wavelengths sequentially. Suitably, the fluorescence imaging system is configured so that the camera is synchronised with the pulsed lasers, so that a given frame records only the emission associated with one excitation wavelength. This means that the emission recorded in a sequence of frames varies according to the same sequence as the variation of the excitation wavelength. This approach maximises the field of view which can be detected, at the cost of more complex illumination and the potential for movement of target pathogens between different frames having a greater effect on relative intensity than for simultaneous imaging.

Alternatively, in implementations which flow the sample (discussed below), sequential detection can be achieved by stacking light sheets of different wavelengths in the manner described above, so that the candidate objects encounter different excitation wavelengths in a set order as it flows through the focal volume. The time course of the fluorescence from a candidate object can thus be indicative of emission corresponding to excitation at a particular wavelength. For example, each candidate object may display a repeatable brightening and dimming as it moves from one excitation colour to another excitation colour, so that the time course of the fluorescence can be tied to different colour channels, e.g. blue illumination, then red illumination.

In a particularly preferred implementation, the imaging step involves a hybrid approach in which fluorescence in a first subset of the multiple colour channels is recorded separately and simultaneously, and fluorescence in a second subset of the multiple colour channels is recorded sequentially.

In particular, the imaging step may involve switching between different excitation wavelengths, and detecting fluorescence associated with each of the excitation wavelengths in the multiple colour channels simultaneously. In other words, the method may involve illuminating at a first excitation wavelength and capturing an image of fluorescence produced in multiple colour channels simultaneously, and illuminating at a second excitation wavelength and capturing an image of fluorescence produced in the multiple colour channels simultaneously. Advantageously, this system permits the assessment of FRET between fluorescent probes, providing even further information to assist in characterising the pathogen type. In addition, it allows ratiometric imaging, allowing the use of more probes.

Sample Movement

Preferably, there is relative motion between the sample and imaging optics during use, in order to maximise the number of individual candidate objects measured in a given time, and minimise the chances of counting the same candidate object multiple times.

The relative motion may be achieved by scanning the sample during the imaging step, for example, by mounting the sample on a translatable stage and scanning the stage during imaging, or by moving the objective lens relative to the sample.

However, more preferably, the relative motion is achieved by flowing the sample during the imaging step. Advantageously, flowing the sample can allow faster data acquisition than a stationary or scanned sample, particularly when combined with the wide-field imaging used in the method of the invention. Furthermore, flowing the sample reduces the potential for double-counting of the same candidate object compared to a stationary or scanned sample, leading to more accurate results. Moreover, flowing the sample allows larger volumes of sample to be probed, which is particularly important in the (typical) situation where the concentration of pathogen in the sample is low. This methodology should be contrasted with the method taught in Shialis et al. which detects virions attached to a slide surface without flow, using total internal reflection fluorescence (TIRF) illumination.

It is especially preferred for the imaging step to involve flowing the sample through a channel, preferably a microfluidic channel, and imaging the sample within the channel. In addition to the general advantages already noted above for imaging under flow, the use of a microfluidic channel maximises the overall percentage of the sample which can be imaged, and hence maximises the percentage of pathogens in a sample which are detected. Preferably, flow within the channel is laminar, at least within the region being imaged.

Preferably, the channel dimensions are the same as the field of view of the imaging system. In other words, the cross-sectional area of the channel is less than or (more preferably) equal to the area imaged by the fluorescence imaging system. Advantageously, this potentially allows every candidate object flowing through the channel to be imaged.

Optionally, the relative motion between sample and imaging optics is lateral motion achieved, for example by flowing the sample across the imaging optics. However, whilst lateral motion can increase data acquisition rate, it will also act to smear photons from a given candidate object across a broader detection area on the camera, decreasing the signal to noise of the measurement and complicating calculation of the overall signal intensity. Indeed, above a certain flow rate this smearing effect will be such that the object becomes undetectable above background noise. Furthermore, this smearing increases the size of the fluorescence signal on the camera for a given candidate object, which increases the probability that signal from multiple candidate objects will overlap, complicating the assignment of an intensity value to each individual candidate object and potentially necessitating dilution of the sample (which counterbalances the potential gain in throughput obtained by the relative motion).

Preferably, the relative motion between sample and imaging optics is vertical motion. The present inventors have found that vertical motion between the sample and imaging optics improves throughput whilst also allowing photons from a given candidate object to be focussed on a small detection area, generally smaller than that obtained during lateral motion.

To achieve such vertical motion, the method of the present invention preferably uses a fluorescent imaging system having:
an objective lens, having an associated focal volume and central lens axis,
a fluidic channel (preferably microfluidic channel, having dimensions matched to the fluorescence imaging system as described above) extending through the focal plane along the central lens axis (in a generally vertical orientation through the focal plane);
an excitation light source for illuminating said focal volume; and
a camera for detecting fluorescence collected from the focal volume by the objective lens in the multiple colour channels. In such instances, the sample is flowed through the fluidic channel, vertically relative to the objective lens, and imaged within the channel.

Advantageously, this particular methodology increases the rate and overall number of candidate objects detected, whilst also ensuring that photons from a given candidate object are received around a fixed point on the detector as the object passes through the focal volume, as described above. This results in the fluorescence emission from a given candidate object being focussed on a relatively small detector area, with all of the associated advantages of that discussed above.

As the skilled reader will appreciate, as the candidate object approaches the focal volume it will be out of focus, and thus fluorescence emission will be spread broadly around a central point. As the candidate object enters the focal volume, the fluorescence emission will become sharp, and then begin to spread again around the central point as the object moves out of focus. Suitably, the overall image of such fluorescence will still appear relatively sharp, since the number of photons received from a candidate object transiting the focal volume will be far greater at the central point of the fluorescence signal than the contributions of out of focus fluorescence. This is particularly true for the relatively low numerical aperture lenses preferred for implementation of the present invention. Nevertheless, in a particularly preferred embodiment the potential issues associated with out of focus fluorescence are addressed by providing the excitation light source as a sheet of light illuminated laterally at and parallel to the focal plane of the objective lens. Advantageously, this helps to restrict fluorescence emission to candidate objects which are within the focal volume of the objective lens, and thus decrease background signal.

In implementations in which the excitation light source operates at multiple wavelengths, the light sheets from the different wavelengths may be co-aligned, or may be stacked relative to one another, as described above.

Optionally, the fluidic channel extends through the focal plane substantially parallel to the central lens axis, in other words at 0° relative to the central lens axis. Alternatively, the fluidic channel extends through the focal plane at an angle relative to the central lens axis, such that fluorescence from a candidate object is received within an elongated area on the camera during the movement through the focal volume. Preferably, the angle is a relatively shallow angle. For example, the angle may be no more than 5°, no more than 10°, no more than 20°, no more than 25° or no more than 30°.

In implementations where the fluidic channel extends through the focal plane at an angle, it may be useful to operate the excitation light source in pulses, such that candidate objects are illuminated for a predetermined period during their flow through the focal volume. Advantageously, the excitation light source is pulsed and switches/alternates between different excitation wavelengths. In this way, the images of the candidate object in the different colour channels are slightly offset. By suitably selecting the flow rate, light pulse rate, frame rate and exposure time, the fluorescence from a candidate object in the different multiple colour channels, when overlaid, can take the form of a barcode.

Using this methodology, it is possible to image all of the multiple colour channels in a single frame on the same camera without having to assign different regions of the camera to detect different colours. However, preferably the emission of at least one of the multiple colour channels is detected on a separate pre-determined detector areas (either on the same camera or on different cameras), since this can help to identify the order of the colours in the “barcode”. For example, in one implementation the emission at a first wavelength is recorded on a first detector area, and the emission from the other wavelength(s) is recorded on a second detector area. In this way, the emission at the first wavelength allows the colour ordering of the barcode to be correctly determined. Preferably, the emission at the longest wavelength is recorded on said first detector area, and emission from the lower wavelength(s) is recorded on a second detector area, since this not only permits the correct colour ordering of the barcode to be determined, but also facilitates measurement of FRET.

The excitation light source may comprise one or more (optical) fibre-coupled light sources, such as one or more fibre-coupled lasers. Advantageously, the use of fibre-coupled light sources can permit a relatively compact construction, whilst permitting easy manipulation and alignment of the excitation light path. In implementations configured to provide multiple light sheets of different wavelengths, there may be multiple fibre-coupled light sources each configured to provide one or a subset of the different wavelengths. Preferably, the excitation light source may comprise multiple fibre-coupled light sources which directly illuminate the focal volume (instead of combining the output of the fibre-coupled light sources before illumination). Advantageously, such an approach avoids the complication and expense of trying to couple the output from multiple optical fibres together. In other words, the excitation light source may omit a fibre-optic combiner.

The multiple fibre-coupled light sources are preferably aligned such that their excitation light is directed in the same plane, preferably aligned such that their excitation light is directed in the focal plane of the imaging lens.

In such implementations, the fibre-coupled light sources may be configured so as to emit their excitation light at an angle relative to one another. This may be achieved by distributing the output of the fibre-coupled light sources around the microfluidic channel, for example, by spacing the fibre optic cables around the microfluidic channel at an angle relative to one another, e.g. with two fibre-coupled light sources emitting their light in the same plane at 90° to one another. For example, the fibre-coupled light sources may be aligned such that their excitation light is directed in the focal plane of the imaging lens, and distributed so that there is an angle between the excitation light of different fibre-coupled light sources.

Alternatively, the fibre-coupled light sources may be configured so as to emit their excitation light parallel to one another. In such implementations, it is preferable for the fibre-coupled light sources to be configured so as to emit their excitation in the same direction. To achieve this, the ends of multiple fibre-coupled light sources may be arranged side-by-side in an array, positioned on one side of the microfluidic channel. Such an array may be a horizontal array (as judged relative to the central axis); that is, with the array extending in the x and/or y direction, instead of being “stacked” in the z direction along the central axis. Advantageously, arranging the fibre-coupled light sources in such a manner can permit a more compact design than spacing the ends of the fibre-coupled light sources around the microfluidic channel. In particular, by placing the ends of the fibre-coupled light sources side-by-side it is relatively straightforward to use a shared cylindrical lens to form the excitation light from the fibre-coupled light sources into light sheets which overlap with one another within the focal volume, in a way which is not possible when the ends of the fibre-coupled light sources are angled relative to one another. In such an implementation, the fibre-coupled light sources may be configured in a side-by-side array in the focal plane of the imaging lens.

Particularly preferred are implementations in which the ends of multiple fibre-coupled light sources are arranged side-by-side in an array with a shared cylindrical lens on one side of the microfluidic channel.

Preferably, the microfluidic channel is configured to provide flow parallel to the central axis and the excitation light source is configured to provide excitation light comprising one or more light sheets comprising different wavelengths, wherein the one or more light sheets are directed across the microfluidic channel, most preferably wherein the one or more light sheets are illuminated laterally at and parallel to the focal plane of the imaging lens (perpendicular to the central axis).

More preferably, the microfluidic channel is configured to provide flow parallel to the central axis and the excitation light source comprises multiple fibre-coupled light sources configured to provide excitation light at different wavelengths, wherein the ends of the fibre-coupled light sources are arranged side-by-side in an array on one side of the microfluidic channel, and wherein a shared cylindrical lens is positioned in front of the ends of the fibre-coupled light sources to shape the excitation light from the multiple fibre-coupled light sources into light sheets during use. Such light sheets are preferably focussed at the centre of the focal volume of the imaging lens.

Further details about preferred implementations of the light sheet illumination can be found in the applicant's earlier application PCT/EP2021/064400.

Light Source Homogenisation

The intensity profile of the external light source may vary across the focal plane, in which case the intensity of detected candidate objects may depend on the position of the candidate object within the external light source beam. This may be corrected for by determining the intensity profile of the external light source and applying a correction to the measured intensity data based on the position of the candidate object.

Most preferably, however, the imaging system may include a light source homogeniser (beam homogeniser). The light source homogeniser smooths out (reduces) variations and irregularities in the intensity profile of the external light source. This minimises or avoids the dependency of detected intensity of a candidate object on the object's position within the external light source beam.

In implementations involving the use of a (micro)fluidic chip including a (micro)fluidic channel, the light source homogeniser may be provided on a region of the (micro)fluidic chip. Preferably, the light source homogeniser may take the form of an optical diffuser on the (micro)fluidic chip, through which the external light source passes before illuminating the focal volume. This optical diffuser may take the form of an etched or roughened surface of the (micro)fluidic chip itself, e.g. achieved by etching or sanding an external surface of the (micro)fluidic chip. Preferably, the external light source is one or more light sheets, and the light source homogeniser is an etched region provided on the (micro)fluidic chip. The etched region may provide a series of etched lines. The lines may be randomly arranged/oriented relative to one another, or alternatively may be provided in a regular array, e.g. horizontal lines, vertical lines, or cross-hatched lines. Preferably, the etched region provides a series of randomly oriented lines.

Alternatively, or additionally, the light source homogeniser may take the form of a lens array homogeniser (e.g. a microlens array homogeniser) or a light pipe homogeniser.

Imaging Calibration

Optionally, the imaging system includes one or more sensors configured to measure characteristics of the system.

For example, the imaging system may include a light sensor to measure power of the excitation light and/or a temperature sensor to measure temperature of the imaging system and/or sample.

In imaging systems incorporating a (micro)fluidic channel, the system may include one or more (optionally all) of the following:
i. a flow sensor to measure flow rate through the (micro)fluidic channel;
ii. a light sensor (such as a photodiode) to measure power of the excitation light before transiting the (micro)fluidic channel;
iii. a light sensor (such as a photodiode) to measure power of the excitation light after transiting the (micro)fluidic channel;
iv. a temperature sensor to measure temperature of the detection system;
v. a temperature sensor to measure temperature of the sample;
vi. a pressure sensor to measure the amount of pressure applied at a sample inlet and/or sample outlet connected to the (micro)fluidic channel.

Preferably, the imaging system includes a feedback system configured to take data from one or more of the above sensors and adjust the operating characteristics of the imaging system accordingly. For example, the imaging system may incorporate any one or more of the following:
a. a feedback system configured to obtain flow rate data from the flow sensor, and adjust the pressure source based on this data until a target flow rate is achieved;
b. a feedback system configured to obtain the detection rate of candidate objects the detection apparatus and flow rate data from the flow sensor, and calculate a concentration of the candidate objects (for example, to check for inhomogeneities in a sample);
c. a feedback system configured to obtain the detection rate of candidate objects from the detection apparatus, and adjust the pressure source based on this data until a target flow rate is achieved;
d. a feedback system configured to obtain a characteristic of the candidate objects from the detection apparatus (for example, total number of candidate objects detected, or total number displaying a specific characteristic), and to adjust operating conditions of the imaging system when a threshold value has been satisfied (for example, decreasing flow rate to look for more weakly fluorescent species);
e. a feedback system configured to obtain a detected characteristic of the candidate objects from the detection apparatus (for example, total number of candidate objects detected, or total number displaying a specific characteristic), and to turn off the pressure source once a threshold value has been satisfied;
f. a feedback system configured to obtain temperature data from the temperature sensor, and adjust the temperature control system until a target temperature is reached;
g. a feedback system configured to obtain excitation light source intensity data from one or more light sensors, and adjust the power of the excitation light source until a target excitation light source intensity is reached;
h. a feedback system configured to obtain signal intensity data from the detector, and adjust the power of the excitation light source until a target signal intensity characteristic is reached (for example, to avoid camera saturation).
i. a feedback system configured to obtain signal intensity data from the detector, and adjust the pressure source until a target signal intensity characteristic is reached (for example, increasing the flow rate reduce time spent in the focal volume and thereby avoid camera saturation).

Optionally, the imaging step includes a calibration step. Such a calibration step may involve carrying out the methods set out above using a calibration sample, to establish any corrections which must be made to the operation of the imaging system (for example, power from the excitation light source) and/or corrections which must be made to analysis of data provided by the detection system. The calibration sample may be, for example, fluorescent beads of a known concentration, size and colour. The calibration step may be used to assess, for example:
the registration between different colour channels of the detection system (to allow the light signals from a candidate objects in different colour channels to be accurately overlaid).
the level of overlap between excitation light provided by different excitation light sources.
inhomogeneities across the detector image arising from inhomogeneities in the excitation light profile (for example, more intense illumination at the middle of the microfluidic channel compared to the sides).
the level of dispersion achieved by a dispersive element.

In methods involving a calibration step, the method preferably involves a data correction step, in which data detected by the detector are corrected to account for the results of the calibration step. For example, the signal intensity data measured by the detector may be adjusted to account for inhomogeneities in the excitation light profile. Additionally or alternatively, the correction step may involve excluding data from regions of the image in which the level of overlap of excitation light is low.

Optionally, the method may involve an adjustment step, in which operating conditions are adjusted based on one or more of the feedback systems mentioned above in relation to the imaging system.

Measurement Step

The measurement step involves detecting the presence of candidate objects in the fluorescence image data which display fluorescence above a threshold, measuring the signal in the multiple colour channels for each of those candidate objects, and recording the signal information as sample data. Preferably, this step involves measuring the signal intensity in the multiple colour channels for each candidate object, and recording the signal intensity information as sample data.

Optionally, the measurement step is carried out after the imaging step. However, preferably, the measurement step is carried out concurrently with the imaging step, to maximise the speed of the method.

The identification of candidate objects and determination of their signal intensity can be carried out using standard image processing procedures used in single particle tracking. Standard software packages are available for such steps, such as tracking software available within the ImageJ software package.

Generally, the signal to noise of the measurement is reasonable since candidate objects bear multiple copies of each of the fluorescent probes, and thus it is straightforward to choose a suitable lower threshold to identify candidate objects. Given that the majority of the image in a given frame is devoid of candidate objects, one option for assigning a suitable lower threshold is to use a set number of standard deviations above the frame's mean pixel count, for example, five standard deviations above the mean pixel count. However, for the sake of minimising computational power, the threshold may be a predetermined value, for example set by the user, based on experience.

The signal intensity of a candidate object may be determined using standard methods.

Preferably, a point spread function is fitted to the signal from each candidate object, and the peak or integrated intensity of the point spread function is recorded. Advantageously, fitting with a point spread function also allows quantification of the size of the candidate object, by measuring the width of the fitted function. Preferably, the fitting is carried out with an elliptical point spread function, to obtain two characteristic dimensions of each candidate object (which may be labelled as the major and minor axes, or height and width). In one implementation, the signal from each candidate object is fitted with a Gaussian function (approximating an Airy disk), and the standard deviation (σ) is recorded as an indication of the dimension of the candidate object. Preferably, the signal is fitted with a two-dimensional Gaussian function. Such a Gaussian takes the general form:

$$f ( x , y ) = A exp ( - ( \frac{{( x - {x}_{o} )}^{2}}{2 {σ}_{X}^{2}} + \frac{{( y - {y}_{0} )}^{2}}{2 {β}_{Y}^{2}} ) )$$

where A is the peak height, x₀ and y₀ are the peak centres, x and y are the spreads about the peak centres, and σₓ and σ_{y} are the standard deviation of the distribution. The standard deviations σₓ and σ_{y} are recorded as two characteristic dimensions of the candidate object. In practice, the axes of the elipse will rarely align perfectly with x and y axes, but instead will be rotated by an angle σ. To account for this, the general form of the Gaussian function fitted to the signal is expressed as:

f(x,y)=A exp(−(x−x₀)²+2b(x−x₀)(y−y₀)(y−y₀)²))

in which:

$$a = \frac{{cos}^{2} θ}{2 {σ}_{X}^{2}} + \frac{{sin}^{2} θ}{2 {σ}_{Y}^{2}}$$

$$b = - \frac{sin 2 θ}{4 {σ}_{X}^{2}} + \frac{sin 2 θ}{4 {σ}_{Y}^{2}}$$

$$c = \frac{{sin}^{2} θ}{2 {σ}_{X}^{2}} + \frac{{cos}^{2} θ}{2 {σ}_{Y}^{2}}$$

and the matrix:

$$[ \begin{matrix}a & b \\ b & c\end{matrix} ]$$

is positive-definite.

Alternatively or additionally, the signal intensity of a candidate object is determined by overlaying a pixel window (in other words, a detector region of a predetermined size) around each candidate object, and summing the pixel values within that pixel window. For example, the signal intensity may be calculated by selecting a square area around the brightest pixel value for each candidate object, and summing the pixel values within that area. The pixel window may correspond, for example, to a square in which a set percentage of the photons from a point source would fall, for example, 95%, 90%, 80%, or 70%. In instances in which there is relative lateral movement between sample and imaging optics (for example, with lateral flow) the pixel window may take some other shape (such as a rectangle) to account for movement of candidate objects across a frame. Advantageously, this pixel window approach is computationally less expensive than fitting a point spread function, and provides good levels of accuracy in the preferred implementations of the present invention where the fluorescence imaging system is configured such that the fluorescence from each candidate object falls on only a small detection area (that is, on a small number of pixels). In such implementations, the measurement may also determine a size of each candidate object, for example by assigning a major and minor axis to the signal within the pixel window, as implemented in the regionprops function of MatLab (wherein the major and minor axis lengths correspond to the major axis and minor axis lengths of an ellipse that has the same normalised second central moments as the detected region).

Optionally, the size measurements are recorded for each of the colour channels. Alternatively, only a single set of size measurements are recorded for a given candidate object. The single set of size measurements may be that of a chosen colour channel. Alternatively, it may be a calculated value based on the sizes calculated across multiple or all colour channels, for example, the mean value across multiple or all colour channels.

Generally, the calculation of the signal intensity involves subtraction of a background signal.

Generally it is preferable to convert the signal intensity values to normalised signal intensity values. In particular, it is preferable to convert the signal intensity values into a photon rate (in other words, the number of photons per second). This may be achieved by dividing the measured signal intensity in a given frame by the exposure time of that frame. This helps to normalise the intensity data to aid analysis in the characterisation step. The normalised signal intensity value may additionally or alternatively be corrected to account for the number of fluorophores present on the different fluorescence probes (as described above) and/or the power density of the illumination. Note that in the discussion below and above the term “signal intensity” is used to refer to both the “raw” signal intensity values or normalised signal intensity values (in particular, photon rates), unless the context requires or specifies otherwise.

Preferably, the measurement step further involves carrying out particle tracking analysis to identify the presence of the same candidate object across multiple frames. Advantageously, carrying out particle tracking analysis minimises the possibility of carrying out double-counting of the same candidate object. Again, the skilled reader will be familiar with standard particle tracking software which can be used in this step.

In instances where a candidate object is tracked across multiple frames, the measurement step may involve calculating only a single associated signal intensity value in each colour channel for that candidate object (in other words, although the candidate object will display variation in signal intensity values across different frames, only one value in each colour channel is recorded for the candidate object). Optionally, the signal intensity value recorded for each colour channel is measured for the frame having the maximum signal intensity value in that colour channel across said multiple frames (even if the maximum signal in the different colour channels occurs within different frames). Alternatively, signal intensity is taken as an average signal intensity value across multiple frames (for example, all frames in which the candidate object is detected). Advantageously, these approaches avoid double-counting of the candidate object, and thus can provide a more accurate estimate of the total number of fluorescence probes of each colour, better accounting for the potential for imperfect alignment of different excitation sources. Similarly, the method may involve recording only a single set of size measurements for each candidate object (instead of recording the size of the object in each tracked frame). The recorded size measurement may be measured for the frame having the maximum signal intensity value in that colour channel across said multiple frames (even if the maximum signal in the different colour channels occurs within different frames), or may be the average across multiple (e.g. all) tracked frames. As described above, the recorded set of size measurements may be based on only one colour channel, or may be calculated based on the results for multiple or all of the colour channels

In implementations involving particle tracking analysis, the measurement step preferably involves calculating a diffusion coefficient for (all, or a subset of) candidate objects. In such implementations, the measurement step may involve calculating a hydrodynamic radius for candidate objects (all, or a subset) based on the diffusion coefficient, sample viscosity (known or estimated) and temperature. Advantageously, use of the hydrodynamic radius instead of the diffusion coefficient removes the effect of temperature and sample viscosity. Advantageously, diluting the sample prior to measurement can simplify calculation of the hydrodynamic radius by minimising the influence of the viscosity of the bodily fluid itself—for example, it may allow the sample viscosity to be estimated to be that of the diluent with minimal loss in accuracy. The hydrodynamic radius may be calculated based on each colour channel separately. Alternatively, the hydrodynamic radius may be calculated based on only one colour channel, or taken as an average across colour channels.

In implementations in which the sample undergoes laminar flow during imaging, the diffusion coefficient is (again) preferably calculated for (all or a subset of) candidate objects. In implementations involving vertical flow described above, the lateral diffusion of the particle can be measured using the same methodology as one would use in the absence of flow. In instances where the flow includes a lateral component, this can be factored into the analysis.

Alternatively, although not essentially, in implementations in which the sample is flowed, the particle tracking may be facilitated by carrying out a two-step imaging procedure, involving:

(2A) an imaging step carried out at low or (more preferably) no flow; and

(2B) an imaging step carried out under flow.

The order of these steps is flexible: (2A) can occur before (2B), (2B) can occur before (2A), and the steps can be alternated. Preferably, however, imaging step (2A) occurs first, as it is relatively straightforward to carry out the low or no flow imaging before initiating flow, rather than trying to reduce flow part-way through implementation. Furthermore, in instances in which data analysis is carried out in parallel with the imaging step, carrying out step (2A) first allows the relatively more data intensive particle tracking analysis to be initiated early in the method, which can lead to faster results overall. Preferably, both steps (2A) and (2B) involve the determination and recording of signal intensity values, as described above.

Sample Data

The “sample data” corresponds to signal information extracted from the fluorescence image data, which is used for subsequent analysis, potentially together with more general information about the sample.

The sample data may comprise a plurality of feature vectors, each feature vector recording characteristic values measured for an individual candidate object during the measurement step.

The feature vector is an n-dimensional vector, in which each dimension corresponds to a characteristic value associated with the candidate object.

For example, in instances where signal intensity values from a candidate object are recorded in 3 different colour channels, the feature vector may include at least 3 dimensions for the signal intensity, each dimension corresponding to the signal intensity (preferably photon rate) in a different colour channel.

In instances where illumination is carried out at M different excitation wavelengths with detection in N different colour channels, the feature vector may include up to M×N dimensions of signal intensity values, corresponding to signal intensity (preferably photon rate) values for all N different channels recorded for each of the M different excitation wavelengths. Advantageously, this feature vector contains the full information on the amount of staining of each fluorescent probe used, but additionally can provide information on the proximity of the labels after they are bound on the pathogen via FRET and/or be used for ratiometric imaging. In practice, however, one or more of the dimensions may not contain useful information, and may be omitted (for example the blue fluorescence detection channel should not produce any meaningful and useful fluorescence signal when the sample is illuminated only with lower energy red light).

The feature vector for a candidate object may also include one or more dimensions recording the diffusion characteristics of the object. For example, the feature vector may include the diffusion coefficient of the object or, more preferably, the object's hydrodynamic radius. Advantageously, the use of a feature vector including diffusion characteristics alongside signal intensity data can increase the versatility and sensitivity of the technique, since the diffusion characteristics will vary from pathogen to pathogen dependent on factors such as the size and shape of the pathogen.

Additionally, the feature vector for a candidate object may include one or more dimensions recording the size of the object, as detailed above. Preferably, the size data records two dimensions, which can be referred to as a minor axis and a major axis.

Optionally, the feature vector is transformed into a modified feature vector. This process transforms the feature vector into more abstract features, often having fewer dimensions. The smaller dimensions of the modified feature vector can aid subsequent analysis in the characterisation step. Various data processing techniques are known to form a modified feature vector whilst minimising loss of information. In particular, the feature vector may be transformed to a modified feature vector using principal component analysis (“PCA”). This converts the original feature vector into a modified feature vector based on linear combinations of the original features. Details of how to carry out PCA can be found, for example, in Joliffe and Cadima, “Principal component analysis: a review and recent developments”, Phil. Trans. R. Soc. A 374: 20150202 (2016). As discussed below, in some cases, a machine learning algorithm may be used to learn how to transform feature vectors into modified feature vectors, in order to facilitate differentiating between different pathogens.

Either the unmodified feature vector or modified feature vector can be used in the characterisation step, and thus henceforward the term “feature vector” will be used to denote both, unless the context specifies or requires otherwise.

Preferably, the feature vector for each candidate object comprises information on signal intensity, diffusion characteristics (preferably hydrodynamic radius) and size (preferably major and minor dimensions), or is a modified feature vector which is derived from information on signal intensity, diffusion characteristics (preferably hydrodynamic radius) and size (preferably major and minor dimensions). Advantageously, the combination of all of these parameters together can help to differentiate pathogen types with great accuracy.

In addition, or alternatively, to the use of feature vectors for each candidate object, the measurement step may involve identifying bounding boxes around each candidate object, and recording the pixel information in each bounding box for subsequent analysis, in the manner taught in Shiaelis et al.

In addition to the information described above in relation to specific candidate objects, the sample data for a given run may also include an indication of imaging parameters. For example, the sample data may include an indication of the frame rate and/or exposure time, and/or the power density of the excitation light source, and/or details about relative movement of the sample and imaging optics (for example, flow rate). In instances in which the sample data is stored in a computer file, the imaging parameters may be included as part of a file header.

Characterisation Step

The characterisation step involves analysing the sample data to determine whether the candidate objects are pathogens and if so, the type of pathogen.

Preferably, the characterisation step involves inputting the sample data into a machine learning classification algorithm.

In a preferred implementation, the machine learning algorithm inputs a feature vector for a given candidate object and outputs a classification probability that the given candidate object corresponds to a particular type of pathogen. Preferably, the machine learning classification algorithm outputs a classification probability for each candidate object, providing a probability of a match between one or several pathogen types, or probability that the candidate object is attributable to “background”. For example, the classification probability for a candidate object may indicate 90% probability for SARS-CoV-2, 5% for PR8 influenza, and 5% for background. The “background” label may be resolved into more detail (for example, extracellular vesicle vs protein aggregate). The probability is generally expressed as a probability vector with entries that correspond to the probability of the candidate object belonging to each type (class) of pathogen, or a background class.

The classification algorithm is created by fitting a training dataset using machine learning analysis, to link signal characteristics (for example, signal intensity, hydrodynamic radius, size, or some abstract combination of these features) to pathogen labels. A supervised learning algorithm may be used to fit the training dataset. Preferably, the supervised learning algorithm is a logistic regression algorithm. Details of such approaches are described, for example, in Deep Learning by Ian Goodfellow, Yoshua Bengio, and Aaron Courville (MIT Press, 2016), which is incorporated herein by reference, in particular section 5.7. The logistic regression algorithm may be implemented using the MatLab software package, for example by using the Machine Learning and Deep Learning application package to train the system, and analysing sample data using the mnrfit function (as described, for example at https://uk.mathworks.com/help/stats/train-logistic-regression-classifiers-in-classification-leamer-app.html and https://uk.mathworks.com/help/stats/mnrfit.html).

The training datasets comprise data obtained by subjecting training fluids to the same measurement procedure detailed above for test samples. In the case of multinomial logistic regression the training datasets comprise feature vectors which are labelled to indicate whether they correspond to a pathogen (or background) and, if so, which pathogen.

The training fluids include samples where the ground truth is known—in other words, where it is established whether a pathogen is present and, if it is, the type or types of pathogen.

The training fluids include one or more control samples, which are samples made with bodily fluids which are confirmed to be free of pathogens (for example, confirmed pathogen free by a method such as PCR).

The training fluids should also include samples made with bodily fluids having a known pathogen present.

Preferably, such training fluids include samples made with bodily fluids of uninfected patients that are spiked with a known pathogen. The pathogen is preferably grown in a relevant human cell line, since the type of cell line chosen will affect the glycosylation profile. Preferably, the training fluids include multiple samples spiked with the same type of pathogen having different associated infection profiles, for example varying the cell line or tissue source from which the pathogen is obtained, or the timepoint during infection at which the pathogen was sampled. This is because the infection profile can lead to detectable differences in labelling level of the pathogen, for the reasons detailed in the “sample type” section above.

Preferably, the training fluids include samples made with bodily fluids of infected patients that contain a pathogen whose identity has been verified by one or more orthogonal methods, such as PCR. Again, the training fluids may include multiple samples spiked with the same type of pathogen having a different associated infection profile. For example, the training fluids may include a sample made with bodily fluid from a patient having a flu virus from lung epithelial cells, and a separate sample made with bodily fluid from a patient having the same flu virus but derived from throat epithelial cells.

The training datasets are generally obtained by subjecting the training fluids to labelling, imaging and measurement under comparable conditions as those used for the test sample. By “comparable conditions” we mean conditions in which any variation will not significantly affect the photon rate (nor optionally the hydrodynamic radius). Preferably, the training datasets are obtained using the same procedure and conditions as the test sample.

For example, suitably the labelling step for the training fluids and test sample use the same fluorescent probes and incubation procedure, to ensure comparable results. Similarly, the fluorescence imaging system used for the sample data and training datasets is preferably the same, and operated under analogous (preferably the same) conditions. For example, excitation light sources may be the same type (that is, operating at the same wavelength, and preferably at the same power density, in the same illumination mode, with the same illumination shape and size, with the same relative alignment of different light sources, and same pulse characteristics). Preferably, the imaging optics are the same and operated at the same conditions, although it is acceptable if the training datasets and sample datasets are obtained at different frame rates, since this can be corrected for through the calculation of photon rates, as detailed above. The skilled reader will be able to identify conditions which can be changed, and those which must remain fixed.

Preferably, the characterisation step involves inputting the feature vector for each candidate object into a multinomial logistic regression algorithm, and generating a probability that the candidate object is of a given type. As noted above, the probability is generally expressed as a probability vector with entries that correspond to the probability of the candidate object belonging to each trained class of pathogen, or a background class (as detailed above).

There are well-known methods to calculate uncertainty of the classification output for a feature vector input. One example would be to use the softmax output of the multinomial logistic regression and interpret the values as probabilities.

Preferably, the candidate object is only assigned to a specific pathogen type if the probability of that classification satisfies one or more pre-determined threshold tests. For example, a candidate object probability vector (corresponding to softmax outputs) specificying 0.45 probability for pathogen A, 0.35 probability for pathogen B, 0.20 probability for pathogen C. Given the similar probabilities of pathogen A and pathogen B, there is a degree of uncertainty about the correct classification, and hence it may be preferable to mark the candidate object as “unclassified”.

Suitably, one criterion is that a candidate object must have at least X (for example, X=0.4, 0.5 or 0.6) probability in one class and that X is at least Y times (for example, Y=1.5, 2, 3) greater than the next leading class probability, otherwise the particle is labelled as unclassified. The predetermined threshold criteria may be that the probability of the candidate object being that pathogen type is at least 0.4, with that probability being at least 1.5 times greater than the next leading class.

As an alternative to the multinomial logistic regression method discussed above, in instances where the sample data comprises or consists of bounding boxes, the bounding boxes may be subjected to analysis using a convolutional network (another type of machine learning classification algorithm). Convolutional networks, also known as convolutional neural networks, are a specialised kind of neural network for processing data that has a known grid-like topology—as in 2D image data. Details of how to carry out such processing are provided in Shiaelis et al., and are discussed in Chapter 9 of Goodfellow et al.

The convolutional network may be implemented using MatLab software, using the trainNetwork function, for example as detailed in Shiaelis et al. In the same way as with the multinomial logistic regression methodology indicated above, this method relies on training the algorithm using a training dataset (as described above) before using it to analyse samples of interest.

As an example of possible parameters for the convolutional network, the network may have 3 convolutional layers in total, with kernels of 2×2 for the first two convolutions and 3×3 for the last convolution. The learning rate may be set to 0.01 and the learning schedule rate kept constant throughout the training. The hyperparameters can be kept the same throughout the training process for all models, with mini batch size set to 1000, the maximum number of epochs to 100 and the validation frequency to 20. In the classification layer, the trainNetwork function of MatLab can be used to take the values from the softmax function and assign each input to one of K mutually exclusive classes (different pathogen types, or background) using the cross entropy function for a 1-of-K coding scheme:

loss=Σᵢ₌₁^{N}Σⱼ₌₁^{K}tᵢⱼ ln(yᵢⱼ)

where N is the number of samples, K is the number of classes, tᵢⱼ is the indicator that the i^{th} sample belongs to the j^{th} class, and yᵢⱼ is the output for sample i for class j (corresponding to the value from the softmax function)—in other words, the probability that the network associates the i^{th} input with class j. A stochastic gradient descent with momentum set to 0.9 may be used as the optimiser.

However, the use of convolutional networks is generally less preferred than multinomial logistic regression. In particular, in the logistic regression methodology the parameters are extracted from the fluorescence images based on well-defined fitting procedures, which are based on models of the underlying physics. In contrast in CNN the system has to be trained to understand what a candidate object looks like, and itself devise a way of characterising the position, shape, intensity and motion of those objects without reference to a model grounded in physical reality. This means that logistic regression can use much simpler and smaller models, with fewer parameters, to analyse data, making the analysis simpler to run. This simpler algorithm and fitting of candidate objects before feeding into the system also makes the system less prone to errors, because there is less for the algorithm to work out. For example, there is greater potential for the CNN to confuse certain noise patterns as a true candidate object, and to miscategorise items. In addition, there is also a danger that a CNN can “overtrain” on a training dataset because of the many parameters of the model, meaning it performs badly with new data that it has not seen before (e.g. a relatively “messy” clinical sample, compared to a cleaner training fluid), due to insufficient flexibility to identify similar but not identical, data.

In addition to, or alternatively to, the use of a machine learning classification algorithms, the characterisation may involve using a training dataset to identify one or more characteristic regions in feature space for different pathogen types, and assessing whether the feature vector for a given candidate object in sample data falls within that feature space. For example, if the feature vector consists of only 2-dimensions corresponding to photon counts in each of two colour channels, one may be able to use a training dataset to plot a 2D scatter plot of photon counts for a given pathogen type, and define a boxed region in which a certain percentage of the signal for that pathogen type appear (for example, 80% or more, 90% or more, 95% or more). In other words, the 2D feature space can be partitioned into different regions or boxes for different pathogen types. The analysis can then involve a simple assessment of whether the candidate objects for sample data falls within an identified region, or falls outside (in which case the candidate object is labelled as “unclassified”). For 2- or 3-dimensional feature vectors, this may be achieved by simple visual inspection of a scatter plot. However, this method is generally only appropriate for relatively simple situations, and going off visual inspection alone is a subjective assessment, prone to human error. More importantly, unlike machine learning approaches, this simple approach does not allow for a statistical analysis of the results. Thus, the use of a machine learning classification algorithm in the characterisation step is highly preferred.

Suitably, the characterisation step involves combining the classification results from the individual candidate objects to produce a sample classification.

The sample classification specifies the type(s) of pathogen present in the sample. Suitably, a pathogen type will only be included in the sample classification if the total number of pathogens of that type identified in the data is above a threshold level, for example, at least 10 pathogens, at least 20 pathogens, at least 50 pathogens. Advantageously, this reduces the possibility of analysis artefacts affecting the results data, or of incorrectly classified extraneous objects unduly influencing the results.

Preferably, the sample classification includes the relative percentage of pathogens of each type. In such instances, the sample classification may omit pathogen types having a percentage below a certain value, for example, pathogens accounting for 15% or less, or 10% or less of the overall number of pathogens detected. Again, advantageously, this reduces the possibility of analysis artefacts affecting the sample classification, and simplifies the characterisation.

Preferably, if the sample as a whole has unclassified candidate objects above a certain threshold, the sample classification may be “unclassified”. For example, the sample may be labelled as “unclassified” if more than a predetermined percentage of candidate objects are unclassified, for example, more than 20%, more than 25, or more than 30%.

Results Data

At its broadest, the results data indicates whether pathogens are present in the sample, and indicates the type(s) of pathogen present in the sample, and optionally the infection profile of those pathogens. The pathogen type may be any of those listed above in the “pathogen type” section, or may be assigned as “unclassified” or “background” as described above.

The results data preferably includes an overall sample classification. As described above, the sample classification may indicate the presence of more than one pathogen type, in patients having two or more co-infections.

The results data preferably additionally or alternatively includes classification data for each candidate object. The classification data for each candidate object may be a single label, corresponding to the pathogen type, unclassified or background, as appropriate. However, more preferably, the classification data consists or comprises of a probability vector as described above. The results data may be separate from sample data. Alternatively, the results data also includes the sample data, by forming a vector for each candidate object including both the sample data and results data for that object. For example, the results data for each candidate object may comprise or consist of a vector having a first set of dimensions corresponding to the feature vector (unmodified or modified) used in the characterisation step and a second set of dimensions corresponding to the single label mentioned above or (more preferably) the probability vector.

Additional Steps

The method of the present invention may include the additional steps (5)-(7) discussed below, in any order or combination.

Learning Step

Preferably, the method also involves step (5), a learning step in which the sample data and results data is fed back into the method to refine future analysis.

Preferably, the learning step involves using the sample data and results data to inform formation of modified feature vectors and/or refine the machine learning algorithm (for example, to retrain the machine learning algorithm).

Machine learning algorithms can be run on pooled features from multiple samples. For example, the method may involve accumulating sample data and results data from a number of samples, and then running a machine learning algorithm to create an improved algorithm for converting feature vectors into modified feature vectors. This learning can lead to a refined algorithm for converting unmodified feature vectors into a modified feature vector which achieves maximum separation (as measured in the multidimensional feature space) of the different types of pathogens in a transformed feature space. As a result, a reliability with which pathogens can be classified may be improved. The machine learning step may involve the use of an unsupervised learning algorithm. For example, the machine learning step may involve the use of PCA, which outputs modified feature vectors corresponding to linear combinations of the original feature vector values. Alternatively, the learning step may use autoencoders, which apply non-linear transformations of the feature vector values to form a modified feature vector.

Thus, the method may include:
collecting sample data and results data corresponding to multiple patient samples, wherein the sample data comprises a plurality of feature vectors; and
training an algorithm via a machine learning technique, using the collected sample data and results data;
wherein the algorithm is configured to transform a feature vector into a modified feature vector having a reduced dimensionality compared to the feature vector, such that modified feature vectors associated with different pathogens have a greater separation, for example greater variance along each feature dimension, compared to the corresponding unmodified feature vectors.

In other words, machine learning is used on a pooled set of sample data, to learn vector transformations that, when applied to feature vectors, produce modified feature vectors which increase or maximise vector separation between different pathogens in a transformed feature vector space.

The trained algorithm may then be used in the characterisation step of the method, for transforming the sample data into a modified feature vector, prior to performing the analysis for generating the result data. In some cases, the algorithm for transforming a feature vector into a modified feature vector may form part of the classification algorithm.

As an additional or alternative learning step, the pooled features from multiple samples may be analysed by unsupervised learning of the feature space. For example, the pooled features may be analysed using a clustering machine algorithm, such as an unsupervised clustering machine algorithm. Such an algorithm seeks to identify clusters of datapoints. To this end, the method may involve subjecting the pooled features to analysis with a K-means algorithm to cluster the transformed feature vectors into K clusters. For example, if the original multinomial logistic regression model is set to distinguish between N classes (separate pathogen types or background), then K can be set to equal N+1 so as to seek to identify a single new class that might be present in the field data. This is particularly pertinent if there is a high incidence of unclassified candidate objects or sample, or if it is known that there is a new pathogen in circulation. Candidate objects belonging to this new class would have to be sufficiently separated in (modified) feature space from the existing N classes in order to be identified by this methodology as a separate cluster.

Details of how to carry out create a classification algorithm without ground truth annotations by carrying out:

1. unsupervised learning of the feature space,

2. unsupervised clustering machine learning analysis

can be found, for example, in Gansbeke et al.

Thus, the method may include:
collecting sample data and results data corresponding to multiple patient samples, wherein the sample data comprises a plurality of feature vectors; and
subjecting the collected sample data and results data to analysis using machine learning, for example, with a clustering machine algorithm.

Optionally, the learning step includes transferring the sample data and results data to a centralised learning database. The centralised learning database is preferably maintained on the internet, to allow users of the method to readily transfer their data, and thus contribute to refinement of the methodology. Advantageously, this can rapidly increase the sensitivity of the characterisation step, particularly for newly encountered pathogens.

In such instances, the results data preferably includes additional information about the test, for example, the date it was carried out, and a relevant geographical location. This can potentially help to track the development of infections.

Suitably, the method involves pooling sample data (and optionally results data) for candidate objects and/or samples labelled as “unclassified”. Advantageously, an accumulation of unclassified candidate objects or samples can be indicative of a new pathogen type. To assist this process, the learning step may involve adding the sample data (and optionally results data) from unclassified candidate objects and/or samples to an unclassified pathogen database (preferably a centralised database). Preferably, data for a given sample is only added to the unclassified pathogen database if a threshold percentage of candidate objects are unclassified, for example, 70% or more, 80% or more, or 90% or more.

Preferably, the pooled sample data (optionally combined with results data) for unclassified candidate objects and/or samples can be subjected to analysis, for example by machine learning. In particular, the pooled data can be used to build a new set of learned features and classification algorithms. This can look for general trends in the pooled data for unclassified candidate objects, to monitor for the emergence of new pathogens. In this regard, the pooled sample data for unclassified candidate objects may be subjected to clustering machine learning analysis, such as a K-means algorithm.

In this regard, as noted above, all candidate objects will be assigned a probability of being a certain pathogen type, and the “unclassified” label will only be applied in the probability does not meet certain threshold conditions. The machine learning can take into account these probability assignments in analysing the data, since this can be indicative of the general nature of the unclassified candidate objects. For example, an accumulation of unclassified candidate objects which most closely resemble SARS-CoV-1 according to the probability assignment can be indicative of the candidate objects corresponding to a new pathogen similar to SARS-Cov-1.

Preferably, the unknown pathogen database can be updated to retrospectively assign a pathogen type (for example, derived from a separate diagnostic test).

Monitoring Step

Preferably, the method also includes step (6), a monitoring step in which the results data is uploaded to a centralised monitoring database. Advantageously, the centralised monitoring database may be used to track incidences of infection, and thus provide a rapid way to identify outbreaks of a particular infection, for example. Furthermore, the centralised monitoring database can be monitored for increases in unclassified particles, or samples that are identified as unclassified, which may be indicative of a new disease type. The centralised monitoring database is preferably maintained on the internet, to allow easy access.

Again, in such instances, the results data preferably includes additional information about the test, for example, the date it was carried out, and a relevant geographical location, to help track the development of infections.

Validation Step

Optionally, the method also includes step (7), a validation step, involving using the results data to identify a suitable secondary assay, and carrying out the secondary assay to confirm the validity of the results data.

This secondary assay may be, for example, PCR, detecting antigens from the pathogen via antibodies or aptamers, or detecting human antibodies against antigens that indirectly indicate the presence of pathogens. As noted in the discussion above, without prior knowledge of the type of pathogen present in a sample, these conventional prior art techniques are difficult to implement—it may be necessary to carry out a whole series of experiments, for example, using a variety of different reagents (for example, different primers, different antibodies). This is time-consuming and expensive, and is particularly problematic for bodily fluids where only a limited amount of sample is available for testing. Advantageously, the method of the present invention can be rapidly run to identify the type of pathogen present, and the validation step can be used to give greater confidence in the diagnosis. In particular, in such instances it may be possible to produce a suitable estimate of the pathogen type rapidly using only a small amount of sample (without having to run to the same level of detail that would be required if identifying the virus based on the method of the present invention alone), allowing the remainder of the sample to be used in the secondary assay.

Furthermore, screening samples initially using the methods of the present invention means that the secondary assay is only implemented for samples that are shown to be worthy of further investigation. This has a number of advantages. Firstly, it means that valuable reagents for the secondary assay are only deployed when warranted. Secondly, in time-critical applications (such as rapid screening of passengers at an airport) the initial results by the method of the present invention can be obtained very rapidly, and the more time-consuming secondary assays which may delay the transit of passengers are only deployed where appropriate. Thirdly, it places less demand on the infrastructure required for the secondary assay (for example, transport of samples and analysis of the sample).

In view of these advantages, the present invention also encompasses a method of carrying out PCR analysis of a pathogen in a bodily fluid, the method involving producing an estimate of the type of pathogen using a method according to the first aspect of the present invention, and selecting a suitable set of primers for PCR analysis based on that estimate.

In addition, the present invention also encompasses a method of identifying a suitable specific label (for example antibody or aptamer) for a pathogen in a bodily fluid, the method involving producing an estimate of the type of pathogen using a method according to the first aspect of the present invention, and selecting a suitable specific label based on that estimate.

Optionally, the validation step may be applied in instances where the output of the assay is unclassified. For example, in instances where the method is used to identify a specific pathogen of interest (such as any one of the specific viruses mentioned in the “pathogen type” section, in particular SARS-CoV-2), if the method detects the presence of pathogens but assigns an unclassified label to those pathogens, the method suitably involves carrying out a secondary assay to test for the specific pathogen of interest (in particular, PCR). Advantageously, this restricts the use of the secondary assay to instances where a clear result is not achieved using the fluorescence based system of the present invention.

Computer-Implemented Systems

In a separate aspect, the present invention provides computer-implemented systems for implementing the methods of the present invention.

Specifically, the invention provides a computer-implemented system for identifying pathogens in a sample, the system being configured to:
train a machine learning algorithm to identify one or more pathogens in sample data, using a labelled set of training data relating to a plurality of candidate objects in training fluorescence image data, the labelled set of training data comprising, for each of the plurality of candidate objects, information indicative of signal intensity (and optionally diffusion characteristics and/or size) in each of multiple colour channels in the training fluorescence image data, wherein each of the multiple colour channels corresponds to a respective fluorescent probe;
receive fluorescence image data corresponding to each of the multiple colour channels;
analyse the received fluorescence image data to detect the presence of a candidate object which displays fluorescence above a threshold, and measure the signal intensity (and optionally diffusion characteristics and/or size) in each of the multiple colour channels for the candidate object to provide sample data for the candidate object; and
use the machine learning algorithm to analyse the sample data so as to generate results data, wherein the results data includes whether the candidate objects are pathogens and, if so, the type of pathogen.

The computer-implemented system may be used, for example, to perform the characterisation step discussed above. Any features discussed above in relation to machine learning algorithms for implementing the first aspect of the invention may be shared with the system of computer-implemented system. For example, the machine learning algorithm of the computer-implemented system may correspond to one of the classification algorithms discussed above.

The training data (or datasets) may be obtained as discussed above in relation to the first aspect, e.g. by subjecting training fluids to a similar measurement procedure to that used for the test samples. For example, the training data may include, for each of the plurality of candidate objects, a signal intensity for that candidate object in each of the multiple colour channels, together with a label indicating whether that candidate object corresponds to a pathogen and, if so, which pathogen. The training data may take any suitable form. In one example, the training data may comprise a labelled feature vector for each candidate object, including a respective dimension for the signal intensity in each of the multiple colour channels.

Each of the multiple colour channels corresponds to a respective fluorescent probe. In other words, a respective fluorescent probe is detectable in its corresponding colour channel. In this manner, the system can determine the relative intensities of the fluorescence signals corresponding to each fluorescent probe for a given candidate object. As discussed above, the relative intensities of signals for the different fluorescent probes provide a fluorescence “signature” for the candidate object, thus enabling it to be identified (e.g. as a specific type of pathogen).

Preferably, each of the multiple colour channels corresponds to a respective fluorescent probe which is configured to bind to a different part or structure of a pathogen. For example, a first colour channel may correspond to a first fluorescent probe which is configured to bind to a surface (or external) structure of the pathogen, such as a surface carbohydrate or a membrane of the pathogen. A second colour channel may correspond to a second fluorescent probe which is configured to bind to an internal structure of the pathogen, such as a nucleic acid of the pathogen. Further colour channels may be used, corresponding to fluorescent probes that bind to other parts of the pathogen.

In one example, the colour channels may correspond to fluorescent probes from the categories discussed above in relation to the first aspect. Thus, the multiple colour channels may correspond to two or more of the following fluorescent probe categories:
(a) a fluorescent probe for binding to a pathogen surface carbohydrate;
(b) a nucleic acid stain;
(c) a membrane stain;

wherein each category of fluorescent probe is detectable in a different one of said multiple colour channels of the fluorescence imaging system.

Optionally, computer-implemented system may further be configured to:
pool sample data and results data corresponding to samples from multiple patients; and analyse the pooled data.

Optionally, the step of analysing pooled data involves subjecting the sample data and results data to a machine learning algorithm to create an algorithm for converting feature vectors into modified feature vectors. This may be achieved using the methodologies taught above, including PCA and autoencoders.

Optionally, the step of pooling sample data and results data involves pooling sample data for unclassified candidate objects (preferably, only for samples having unclassified candidate objects above a threshold amount, as taught above), and running the pooled sample data for unclassified candidate objects through a classification algorithm as taught above (optionally, the same classification algorithm used for classification of individual candidate objects).

Preferably, the machine learning algorithm employs multinomial logistic regression so as to assign a probability that each candidate object is a pathogen of a given type. The algorithm generally assigns an “unclassified label” if the probability does not meet some threshold condition (as discussed above).

Preferably, the invention includes the further step of pooling sample data and results data for unclassified candidate objects (for example, by adding the information to a database, preferably a centralised database containing data from multiple different experiments), and subjecting the pooled data to a machine learning algorithm to analyse the unclassified candidate objects.

In a further aspect, the invention provides a computer-implemented system for identifying pathogens in a sample, the system being configured to:
train a machine learning algorithm to identify one or more pathogens in sample data, using a labelled set of training data comprising information indicative of signal intensity (and optionally diffusion characteristics and/or size) in each of multiple colour channels for candidate objects in fluorescence image data;
receive fluorescence image data obtained for the sample, corresponding to each of the multiple colour channels;
analyse the received fluorescence image data to detect the presence of a candidate object which displays fluorescence above a threshold, and measure the signal intensity (and optionally diffusion characteristics and/or size) in the multiple colour channels for each candidate object to provide sample data for the candidate object;

use the machine learning algorithm to analyse the sample data so as to generate results data, wherein the results data includes whether the candidate objects are pathogens and, if so, the type of pathogen;
pool sample data and results data corresponding to multiple patient samples;
analyse the pooled data.

The computer-implemented system of this aspect may include any of the features discussed above in relation to the computer-implemented system of the previous aspect. For example, the training data may be as discussed above. The steps of pooling sample data an analysing the pooled data may be performed as discussed above.

In a further aspect, the present invention provides a computer processor configured to carry out the methods of the present invention.

For example, the invention provides a computer processor configured to:
train a machine learning algorithm to identify one or more pathogens in sample data, using a labelled set of training data relating to a plurality of candidate objects in training fluorescence image data, the labelled set of training data comprising, for each of the plurality of candidate objects, information indicative of signal intensity (and optionally diffusion characteristics and/or size) in each of multiple colour channels in the training fluorescence image data, wherein each of the multiple colour channels corresponds to a respective fluorescent probe;
receive fluorescence image data obtained for the sample, corresponding to each of the multiple colour channels;
analyse the received fluorescence image data to detect the presence of a candidate object which displays fluorescence above a threshold, and measure the signal intensity (and optionally diffusion characteristics and/or size) in each of the multiple colour channels for the candidate object to provide sample data for the candidate object; and
use the machine learning algorithm to analyse the sample data so as to generate results data, wherein the results data includes whether the candidate objects are pathogens and, if so, the type of pathogen.

In addition, the invention provides a processor configured to:
train a machine learning algorithm to identify one or more pathogens in sample data, using a labelled set of training data comprising information indicative of signal intensity (and optionally diffusion characteristics and/or size) in each of multiple colour channels for candidate objects in fluorescence image data;
receive fluorescence image data corresponding to each of the multiple colour channels;
analyse the received fluorescence image data to detect the presence of a candidate object which displays fluorescence above a threshold, and measure the signal intensity (and optionally diffusion characteristics and/or size) in the multiple colour channels for each candidate object to provide sample data for the candidate object;
use the machine learning algorithm to analyse the sample data so as to generate results data, wherein the results data includes whether the candidate objects are pathogens and, if so, the type of pathogen;
pool sample data and results data corresponding to multiple patient samples; and
analyse the pooled data.

Kit of Parts

In a separate aspect, the present invention provides a kit of parts for carrying out the method of the present invention.

The kit of parts comprise at least two of the above fluorescent probe (a), fluorescent probe (b) and fluorescent probe (c). Preferably, the kit of parts comprises all of fluorescent probe (a), fluorescent probe (b), and fluorescent probe (c). The fluorescent probes may be included together in a package, optionally including instructions on their use. The kit of parts may include additional reagents for sample preparation, for example, a buffer optionally containing the additional components mentioned in the “sample preparation” section. Optionally, the kit of parts comprises the at least two fluorescent probes included a shared diluent.

The particular combination of fluorescent probes (a)-(c) may be one of those indicated in the “combination of fluorescent probes” section above.

Preferred Implementations

In a particularly preferred implementation, the invention provides a method of identifying pathogens (preferably, viruses, in particular SARS-CoV-2) in a sample of bodily fluid using a fluorescence imaging system configured to detect fluorescence in multiple colour channels, wherein the fluorescence imaging system comprises:
an objective lens, having an associated focal volume and central lens axis;
a (micro)fluidic channel extending through said focal volume along said central lens axis (in a generally vertical orientation through the focal plane);
an excitation light source for illuminating said focal volume; and
a camera for detecting fluorescence collected from the focal volume of the objective lens in said multiple colour channels;
preferably wherein 90% of the point spread function of the system at the focal plane of the objective lens occupies a camera area corresponding to 49 pixels or fewer (7×7), 36 pixels or fewer (6×6), 25 pixels or fewer (5×5), 16 pixels or fewer (4×4), 9 pixels or fewer (3×3) or 4 pixels (2×2);
the method comprising:
(1) a labelling step, involving incubating the sample with fluorescent probe (a) and at least one of fluorescent probes (b) and (c);
(2) an imaging step, involving flowing the sample through said fluidic channel (vertically through the focal volume) and imaging the sample within the channel using the fluorescence imaging system to produce fluorescence image data for each of the multiple colour channels, preferably using sequential illumination at different excitation wavelengths;
(3) a measurement step, involving detecting the presence of candidate objects in the fluorescence image data which display fluorescence above a threshold, and recording the signal (preferably intensity, diffusion characteristics and size) in the multiple colour channels for each candidate object as sample data; and
(4) a characterisation step, involving inputting the sample data into a machine learning classification algorithm to generate result data, wherein the result data includes whether the candidate objects are pathogens and if so, the type of pathogen.

Preferably, the flow during the imaging step is parallel to the central lens axis of the objective lens such that fluorescence emission from a candidate object is received around a fixed point on the detector as the object passes through the focal volume.

In an especially preferred implementation, the excitation light source comprises a sheet of light illuminated laterally at and parallel to the focal plane of the objective lens, preferably having a thickness comparable to the focal volume of the objective lens. Advantageously, this can improve signal to noise of the technique and facilitate calculation of signal intensity associated with a pathogen by restricting fluorescence emission to pathogens which are within the focal volume of the objective lens.

In such implementations, the excitation light source preferably comprises a stack of light sheets of different wavelengths. Optionally, the light sheets are ordered to match the position of the focal depth of the lens at different wavelengths (for example, if the focal depth for blue wavelengths appears below red wavelengths, then the light sheets are stacked blue below red).

In this way, candidate objects will encounter different excitation wavelengths as they flow through the focal volume of the lens.

Preferably, the excitation light sources are operable at wavelengths of 488 nm, 561 nm, 640 nm, and optionally 750 nm. In such embodiment, the probes are preferably one of the specific combinations 1 or 2 set out above.

In an especially preferred implementation the machine learning classification algorithm outputs a classification probability for each of the possible pathogen types that a candidate object might correspond to.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 is a schematic showing sample preparation and labelling according to the method of the invention;

FIG. 2 is a schematic showing a preferred method for carrying out the imaging step of the invention;

FIG. 3 is a schematic showing detection of fluorescence from a virus particle detected using the apparatus of FIG. 2 across a series of camera frames obtained upon illumination with a blue laser (left-hand image), green laser (central image) and red laser (right-hand image);

FIG. 4 is a schematic showing detection of fluorescence from a virus particle detected using alternative apparatus in which the microfluidic channel depicted in FIG. 2 is tilted relative to the central axis of the objective lens;

FIG. 5 is a schematic showing detection of fluorescence from a virus particle detected using alternative apparatus in which the microfluidic channel depicted in FIG. 2 is tilted relative to the central axis of the objective lens, and the image splitter is omitted;

FIG. 6 is a hypothetical feature vector representing the virus particle depicted in FIG. 3;

FIG. 7 is a hypothetical modified feature vector obtained by subjecting the feature vector of FIG. 6 to a transformation based on an algorithm derived from principal component analysis;

FIGS. 8A-8D are scatter plots of blue photon counts versus red photon counts for a raw saliva sample either containing no spiked virus (FIG. 8A) or containing a spiked virus corresponding to PR8 (FIG. 8B), NL63 (FIG. 8C) or a combination of both PR8 and NL63 (FIG. 8D).

FIG. 9 is a plot showing linear scaling between the number of pathogens detected in experimental example 1 with increasing concentrations of influenza PR8 strain (with error bars corresponding to standard deviation across.

FIG. 10 is a flowchart showing the steps carried out by a processor implementing an identification method according to the present invention.

FIG. 11 is a schematic showing the provision of an optical diffuser as part of a fluidic channel, to achieve light beam homogenisation.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

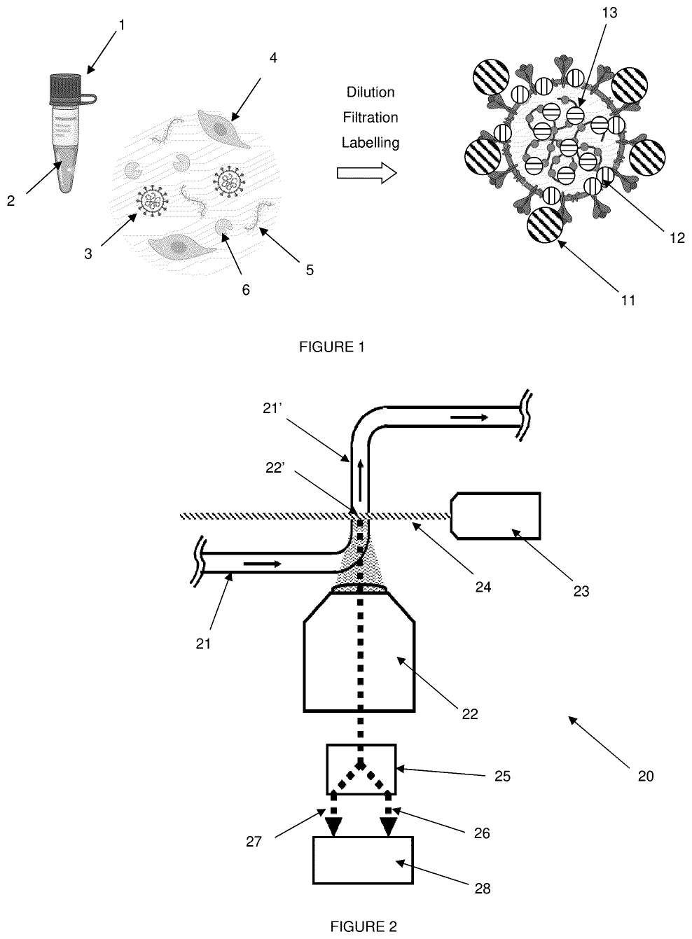

For the purposes of better understanding the invention, the following description and accompanying schematics in FIGS. 1 to 7 and 10 provide an example of methods according to the present invention:

In a first step, a human test subject displaying symptoms of a viral infection deposits saliva sample 2 into a collection tube 1, which is subsequently sealed before analysis. In this case, the saliva contains virions 3 alongside endogenous components such as cells 4, mucin 5 and enzymes 6 (note that the various components are not to scale—the virion 3 will be much smaller than cell 4, for example). In this case, the virions 3 correspond to SARS-CoV-2.

Next, 2 μl of saliva sample 2 are mixed with 18 μl of a labelling solution consisting of 40 nM of fluorescently labelled lectin 11, 1 μM of a membrane stain 12 and 5 μM of a nucleic acid stain 13 in a HEPES buffer containing 150 mM NaCl at pH 7.5. In this case, the fluorescently labelled lectin 11 is Galanthus nivalis lectin (GNA) labelled with Cy5 having a fluorescence emission spectrum in the red region; the membrane stain 12 is Dil having a fluorescence emission spectrum in the orange region; and the nucleic acid stain 13 is Syto13 having a fluorescence emission spectrum in the green region. After 3 minutes of incubation, the fluorescently labelled lectin 11 has bound to mannose motifs on SARS-CoV-2 spike protein, the membrane stain 12 has bound to the membrane, and the nucleic acid stain 13 has permeated the virus membrane and bound to the single-stranded RNA inside the membrane, as depicted in the righthand part of FIG. 1.

Imaging of the labelled sample is then carried out using fluorescence imaging system 20 shown in FIG. 2. The fluorescence imaging system 20 comprises a fluidic chip incorporating a microfluidic channel 21 having a section 21′ running vertically along the central lens axis of a 20× magnification 0.45 numerical aperture objective lens 22. The diameter of the microfluidic channel 21 is slightly less than the width of the focal volume 22′ of objective lens 22. The system 20 incorporates a laser system 23 capable of emitting a light sheet 24 which passes through the focal volume 22′. The thickness of the light sheet 24 is approximately 10 μm which is the same as the thickness of the focal volume 22′.

In this case, the laser system 23 is capable of providing alternating pulses of light at different wavelengths, switching between a blue laser, green laser and red laser. Fluorescence emission arising within the focal volume 22′ is collected by objective lens 22 and fed to an image splitter 25, which uses a longpass dichroic mirror (not shown) to separate red emission 26 from green/orange emission 27. The red emission is then directed to one half of camera 28, and the green/orange emission is directed to the other half of the camera 28.

The intensity profile of the light sheet 24 from the blue, green and red laser is homogenised through use of an optical diffuser. The optical diffuser is shown in FIG. 11. Here the laser light 24 is provided by optical fibre 111, and passes through cylindrical lens 113 to form a light sheet which illuminates microfluidic channel 21. The exterior surface of the microfluidic channel includes an optical diffuser 115, corresponding to lines etched into the surface of the microfluidic channel in a random configuration. The lines help to homogenise the light profile such that the illumination intensity is smoothed across the field of view of the objective lens.

During imaging, the test sample is flowed into the microfluidic channel 21 at a rate sufficient to allow a full cycle between blue, red and green lasers to be implemented whilst a virus is within the focal volume. The frame rate and exposure time of the camera are chosen so that each frame corresponds to emission stimulated by only one laser. In this case, the video frames can be grouped into blocks of three, with the first frame corresponding to illumination by the blue laser, the second frame corresponding to illumination by the green laser, and the third frame corresponding to illumination by the red laser.

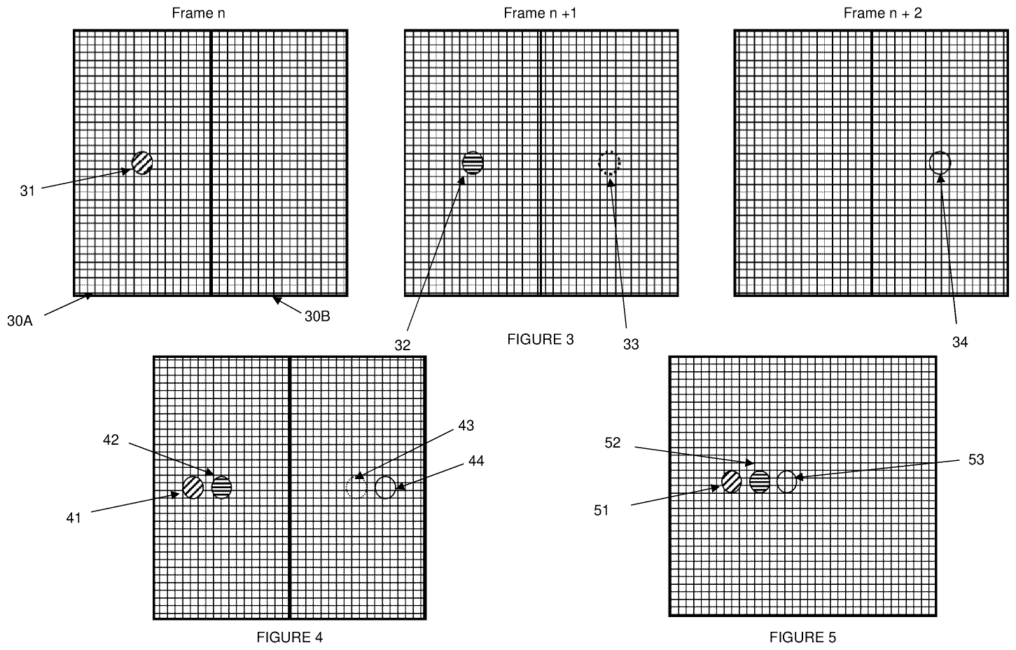

FIG. 3 shows the detected images across a block of three frames from a virus labelled with all three labels 11, 12 and 13. Each image has two halves—a first region 30A which detects green/orange emission, and a second region 30B which detects red emission. The left-hand frame (labelled n) shows fluorescence emission 31 detected upon excitation of nucleic acid stain 13 with a blue laser. The central frame (labelled n+1) shows emission detected upon excitation with the green laser. In this case, green fluorescence 32 from membrane stain 12 is detected in region 30A, and a fainter red fluorescence 33 is detected from fluorescently labelled lectin 11. In this case, the detection of fluorescence 33 is indicative of FRET occurring between a portion of the membrane stain 12 and fluorescently labelled lectin 11. Finally, the right-hand frame (labelled n+2) shows emission detected upon excitation with the red laser, leading to the detection of fluorescence 34 from fluorescently labelled lectin 11. In all cases, the fluorescence emission is focussed onto a relatively small number of pixels, so that the emission is consistently captured within a 7×7 pixel region. Moving from frame n, to n+1 and n+2 the pathogen is flowing vertically away from the objective lens, but the combination of laminar flow and image capture characteristics (frame rate and exposure time) are such that the particle remains in focus throughout. In this case, the detection of the same pathogen in multiple frames allows particle tracking to be carried, which is used to calculate a diffusion coefficient for the pathogen, and from that a hydrodynamic radius. Furthermore, the size of the pathogen is calculated based on fluorescence from the membrane stain.

FIG. 4 shows detected images from an alternative implementation in which microfluidic channel 21 is oriented at an acute angle relative to the central axis of objective lens 22, and the blue, green and red lasers are pulsed over the course of a single frame with gaps between each laser's operation. In this case, the fluorescence resulting from green, orange and red fluorescence is spread laterally across the detector. The lateral flow of material occurs from left to right on the figure, so that green fluorescence 41 is first detected upon excitation with the blue laser, orange fluorescence 42 and its associated FRET signal 43 are then detected further rightwards on the detector upon excitation with the green laser, and red fluorescence 44 is then detected further rightwards still upon excitation with the red laser.

FIG. 5 shows an alternative implementation, in which the fluorescence system described above in relation to FIG. 4 is further modified to remove the image splitter. In the same way as for FIG. 5, the green fluorescence 51, orange fluorescence 52 and red fluorescence 53 are laterally spaced across the frame due to the combination of the lateral flow component and alternating laser excitation. However, in this case information about the FRET from the membrane stain 12 to fluorescently labelled lectin 11 is lost.

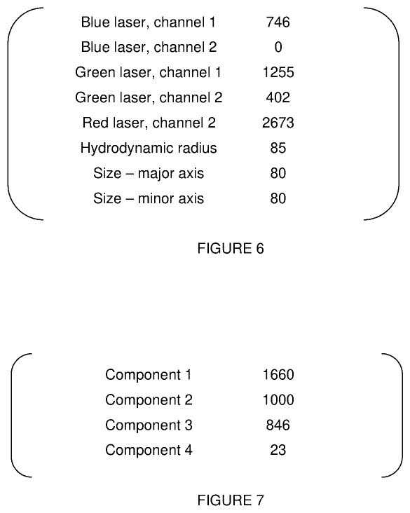

FIG. 6 shows a representation of an 8-dimensional feature vector for the virus shown in FIG. 3, in which “channel 1” corresponds to the green/orange emission channel and “channel 2” corresponds to the red emission channel. The vector reports photon rates obtained by integrating the detected fluorescent intensity above background for fluorescence 31, 32, 33 and 34, and normalising to give the photons detected per second. The entry for “green laser, channel 2” shows the FRET from the membrane stain 12 when illuminated with the green laser. In this case, the signal for the red laser in channel 1 is not included in the feature vector, since it will not convey any meaningful information in this instance. The feature vector also includes dimension information, including the hydrodynamic radius measured by particle tracking, and the size of the particle (corresponding to the cyx and σ_{y} from fitting the particle to a 2D Gaussian function).

Some of the features in the feature vector may be correlated. For example, a sum photon rate in all the channels may be correlated with the diffusion coefficient because larger particles are likely to be brighter. Accordingly, the feature vector can in many cases be transformed into a modified feature vector, which has fewer dimensions compared to the original feature vector, and which facilitates characterisation of the sample.

As an example, FIG. 7 shows a hypothetical modified 4-dimensional feature vector obtained by transforming the 8-dimensional feature vector of FIG. 6 based on principal component analysis. The vector values correspond to linear combinations of the values in FIG. 6 arrived at based on feature learning from previous datasets.

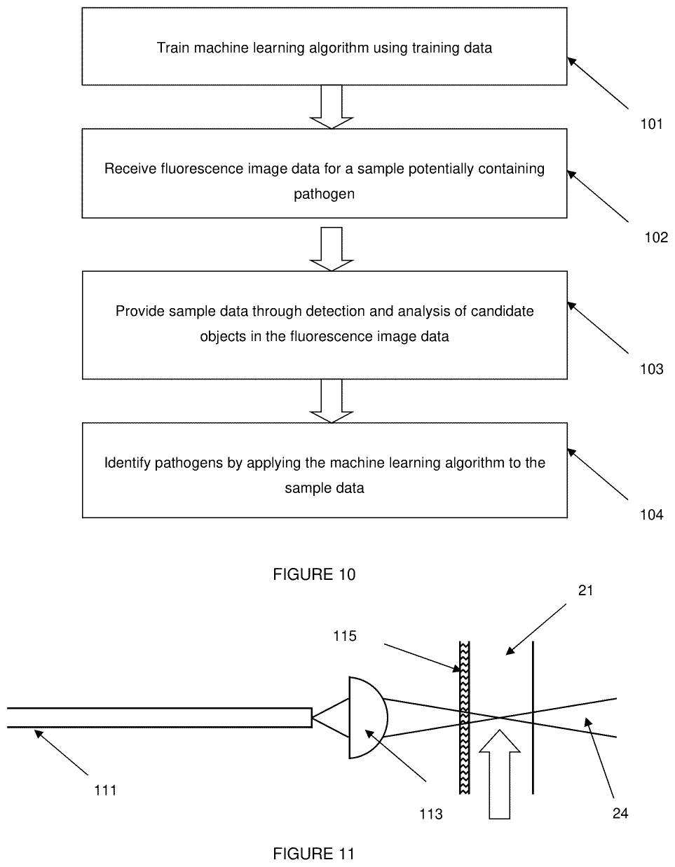

FIG. 10 is a flowchart showing the steps carried out by a processor implementing an identification method according to the present invention. In step 101, a machine learning algorithm is trained to identify one or more pathogens in sample data, using a labelled set of training data comprising information indicative of signal intensity (and optionally diffusion characteristics and/or size) in each of multiple colour channels for candidate objects in fluorescence image data. The training data is obtained from training fluids where the ground truth is known—that is, where it is established whether a pathogen is present and, if it is, the type or types of pathogen. In this case, the training data fed into the learning algorithm corresponds to data obtained for training fluids containing one of SARS-CoV-1, MER-CoV, SARS-CoV-2, HCoV-NL63, HCoV-229E, HCoV-0043 and HCoV-HKU1.

In step 102, the processor receives fluorescence image data obtained from a sample undergoing evaluation, comprising data in multiple colour channels. The fluorescence image data is obtained under the same conditions as those used to obtain the training data.

In step 103, the processor analyses the fluorescence image data to detect the presence of a candidate object which displays fluorescence above a threshold, and measure the signal intensity (and optionally diffusion characteristics and/or size) in each of the multiple colour channels for the candidate object to provide sample data for the candidate object.

Finally, in step 104, the processor uses the trained machine learning algorithm to analyse the sample data so as to generate results data. The results data includes a classification probability that a given candidate object corresponds to a particular type of pathogen, or should be “unclassified” or classed as background. In this instance, based on the training data, the results data determines the classification probability that a given candidate object is one of SARS-CoV-1, MER-CoV, SARS-CoV-2, HCoV-NL63, HCoV-229E, HCoV-0043, or HCoV-HKU1, is unattributable to one of these viruses, or is simply background.

### EXAMPLES

### Example 1

The following example was carried out, to demonstrate the ability of the technique to identify different virus types.

1. Raw saliva was collected from healthy donors (1-2 ml per donor) in a 50 ml centrifuge tube. 100 μl was removed and mixed with 900 μl HEPES-buffered saline+2.5 mM CaCl₂). 100 μl diluted saliva was then passed through a syringe-driven 0.2 μm nitrocellulose filter.

2. Prior to staining, filtered saliva was spiked with PR8 influenza A virus and NL63 alphacoronavirus alone or in combination. In both cases the viruses were derived from in vitro-infected human cell lines and were not fixed prior to use. Virus preparations were prediluted in filtered saliva such that a constant volume of 1 μl virus into 7 μl filtered saliva was always used for spiking.

3. 8 μl of spiked saliva (or unspiked control) were stained by addition of 1 μl 400 nM Wheat Germ Agglutinin-Alexa Fluor 647 conjugate (emitting in the red region) and 1 μl 50 μM Syto13 (emitting in the green region) followed by incubation at room temperature for 5 min.

4. After incubation, stained samples were immediately injected into a microfluidic chip mounted to a Nanoimager machine (ONI, Oxford, UK). Samples were flowed at 0.7 nL/20 ms vertically though the focal volume and excited with sequential 473 and 640 nm lasers for 20 ms/frame. 3000 acquisition cycles were recorded, with images collected simultaneously above and below 640 nm following dichroic spectral separation of the emitted light.

5. Fluorescence signals showing fluorescence in both red and green channels were identified in the collected images.

6. For samples containing only PR8 or NL63, 90% of the detected fluorescence signals were randomly selected, labelled with the pathogen type, and used to train a multinomial logistic regression machine learning algorithm.

7. For the mixed sample, all of the detected fluorescence signals were analysed using the trained algorithm, and classified as either PR8 or NL63, utilising unmodified feature vectors.

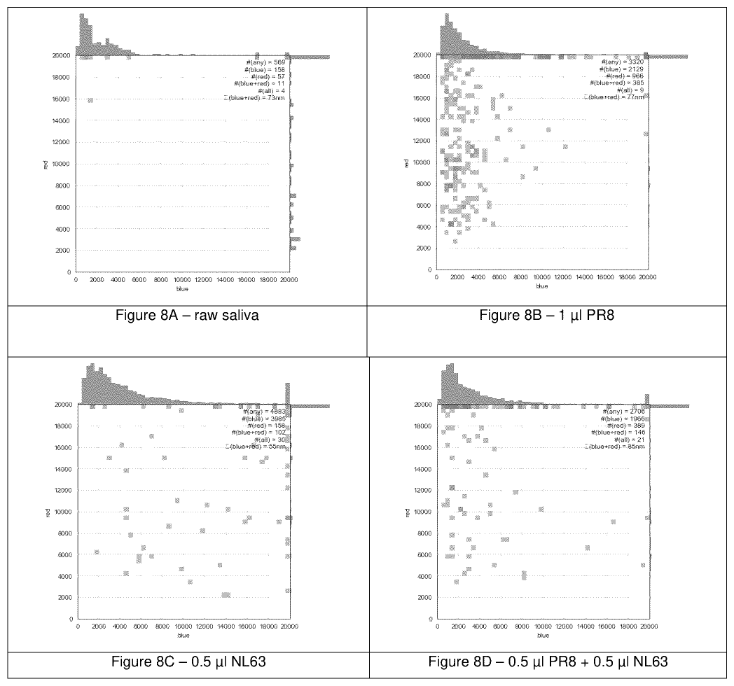

The results of these experiments are shown in FIGS. 8A-8D. The 2D joint histogram displays only particles that are above background threshold in both the green and red channels (single-positive events are not shown). The horizontal and vertical 1D marginal histograms display any particle which was observed under blue illumination and red illumination respectively. Particle counts are given in each plot. Histograms show the intensity distribution of the particles. Axes correspond to photon count in each channel.

From a simple visual inspection of the FIG. 8A it can be seen that the raw saliva sample produced negligible candidate objects displaying fluorescence in both green and red channels. Furthermore, visual inspection of FIGS. 8B-D show a clear difference in the scatter plots for PR8 versus NL63.

As shown in Table 2 below, the analysis identified 1041 PR8 pathogens across three acquisitions of the sample spiked with PR8 alone and 407 pathogens across three acquisitions of the sample spiked with NL63 (based on the ground truth assumption that every dual-labelled candidate object is a pathogen). The mixed pathogen sample separated identified 307 candidate objects classified as PR8 and 129 classified as NL63.

Finally, the remaining 10% of fluorescence signals identified in the individual PR8 and NL63 samples were analysed using the trained machine learning algorithm. The accuracy of the classification was then determined by determining the number of fluorescence signals identified to be the correct virus type (on the ground truth assumption that all fluorescence signals showing both green and red emission were attributable to labelled pathogen).

TABLE 2

| | |
|---|---|
| PR8 particles | NL63 particles |

Single virus samples

| | | |
|---|---|---|
| 1 μL PR8 | 1041 (ground | 0 (ground |
| (3x acquisitions) | truth assumption) | truth assumption) |
| 1 μL NL63 | 0 (ground | 407 (ground |
| (3x acquisitions) | truth assumption) | truth assumption) |
| Single particle classification | 90% | 84% |
| accuracy based on ground | | |
| truth test dataset | | |

Mixed virus sample

| | | |
|---|---|---|
| 0.5 μL PR8 + 0.5 μL NL63 | 307 (classified) | 129 (classified) |
| (3x acquisitions) | | |

The results show that, even with the use of signal intensity values from only two types of label, the method is able to identify particles with very high accuracy. The skilled reader will understand that the numerous layers of additional information that can be derived from more complex implementations of the invention (involving, for example, the use of a nucleic acid stain, an additional carbohydrate probe, and optionally determining other attributes such as diffusion characteristics and size) will result in even higher levels of accuracy.

### Example 2

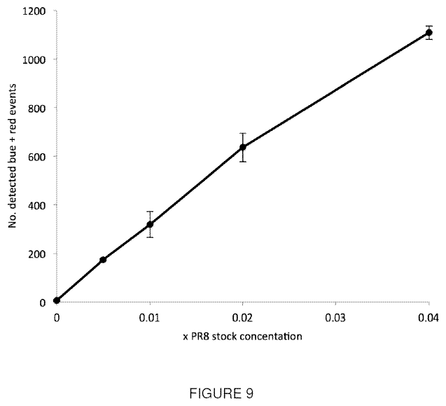

A further example was carried out, to demonstrate the sensitivity of the technique to the level of pathogen. The procedure was carried out as described in Example 1, but using saliva samples titrated with increasing concentration of PR8 influenza A. From FIG. 9, a clear linear correlation is observable between the number of dual positive red and green events and the dilution factor of the PR8 used. The error bars correspond to standard error across 3 repetitions of the experiment.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps. It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### REFERENCES

Demchenko et al. “Monitoring Biophysical Properties of Lipid Membranes by Environment-Sensitive Fluorescent Probes”, Biophys. J. 96(9): 3461-3470 (2009)
Gansbeke et al. “SCAN: Learning to Classify Images without Labels” arXiv:2005.12320, available at https://arxiv.org/pdf/2005.12320.pdf.
Goodfellow, I.; Bengio, Y.; and Courville, A.. “Deep Learning”, MIT Press, (2016)
Joliffe and Cadima, “Principal component analysis: a review and recent developments”, Phil. Trans. R. Soc. A 374: 20150202 (2016)
Shiaelis, N. et al. “Virus detection and identification in minutes using single-particle imaging and deep learning”, MedRxiv, October 2020
ThermoScientific website “Calculate dye:protein molar ratios” at http://tools.thermofisher.com/content/sfs/brochures/TR0031-Calc-FP-ratios.pdf

1. A method of identifying pathogens in a sample using a fluorescence imaging system configured to illuminate the sample with an excitation light source and detect resulting fluorescence in multiple colour channels, comprising:
(1) a labelling step, involving incubating the sample with fluorescent probes from at least two of the following categories:
(a) a fluorescent probe for binding to a pathogen surface carbohydrate;
(b) a nucleic acid stain;
(c) a membrane stain;
wherein each category of fluorescent probe is detectable in a different one of said multiple colour channels of the fluorescence imaging system;
(2) an imaging step, involving imaging the sample using the fluorescence imaging system to produce fluorescence image data for each of the multiple colour channels;
(3) a measurement step, involving detecting the presence of candidate objects in the fluorescence image data which display fluorescence above a threshold, and recording the signal in the multiple colour channels for each candidate object as sample data;
(4) a characterisation step, involving analysing the sample data to generate result data, wherein the result data includes whether the candidate objects are pathogens and if so, the type of pathogen.

2. A method according to claim 1, wherein the imaging is wide-field imaging.

3. A method according to claim 1, in which the labelling step involves incubating the sample with fluorescent probe (a) and at least one of fluorescence probe (b) or (c).

4. A method according to claim 1, in which the labelling step involves incubating the sample with all of fluorescent probes (a), (b) and (c).

5. A method according to claim 1, in which the labelling step involves incubating the sample with multiple fluorescent probes in category (a), targeting different pathogen surface carbohydrates, wherein all of the probes (a), (b) and (c) are detectable in a different one of said multiple colour channels of the fluorescence imaging system.

6. A method according to claim 1, wherein the fluorescent probe (a) is a fluorescently labelled lectin, or a method according to claim 5 wherein each fluorescent probe (a) is a fluorescently labelled lectin.

7. A method according to claim 1, wherein the sample is flowed during the imaging step.

8. A method according to claim 7, wherein the fluorescence imaging system comprises:
an objective lens, having an associated focal volume and central lens axis,
a fluidic channel extending through the focal plane along the central lens axis;
the said excitation light source, for illuminating said focal volume; and
a camera for detecting fluorescence collected from the focal volume by the objective lens in the multiple colour channels;
and wherein the sample is flowed vertically through the focal plane during the imaging step.

9. A method according to claim 8, wherein the excitation light source produces a sheet of light illuminated laterally at and parallel to the focal plane of the objective lens, preferably wherein the thickness of the sheet of light is greater than or equal to the cross-section of the fluidic channel.

10. A method according to claim 9, wherein the imaging step involves alternating the excitation light source between different excitation wavelengths.

11. A method according to claim 1, in which the imaging step involves detecting fluorescence in the individual colour channels simultaneously.

12. A method according to claim 11, wherein the imaging step involves alternating the excitation light source between different excitation wavelengths and detecting fluorescence emission in the different colour channels simultaneously on separate pre-determined detector areas.

13. A method according to claim 1, wherein the characterisation step involves inputting the sample data into a machine learning classification algorithm.

14. A method according to claim 13, wherein the machine learning classification algorithm outputs a classification probability for each of the possible pathogen types that a candidate object might correspond to.

15. A method according to claim 14, wherein the machine learning classification algorithm assigns a specific pathogen type to a candidate object if one or more predetermined threshold criteria are satisfied.

16. A method according to claim 15, wherein the predetermined threshold criteria are that the probability of the candidate object being that pathogen type is at least 0.4, and that probability is at least 1.5 times greater than the next leading class.

17. A method according to claim 15, wherein the candidate object is labelled as an unclassified candidate object if the pre-determined criteria are not satisfied.

18. A method according to claim 17, wherein the sample data and results data for unclassified candidate objects are pooled and subjected to machine learning analysis.

19. A method according to claim 17, wherein the characterisation step involves combining the results data from the candidate objects to produce a sample classification which specifies the types of pathogen present in the sample.

20. A method according to claim 1, wherein the method is used to identify the presence of a virus.

21. A method according to claim 20, wherein the method is used to distinguish between viruses selected from at least two of a coronavirus, respiratory syncytial virus (RSV), influenza, rhinovirus, and adenovirus.

22. A method according to claim 20, wherein the method is used to assess whether a sample contains SARS-CoV-2.

23. A method according to claim 1, for identifying pathogens in a sample of bodily fluid using a fluorescence imaging system configured to detect fluorescence in multiple colour channels, wherein the fluorescence imaging system comprises:
an objective lens, having an associated focal volume and central lens axis;
a (micro)fluidic channel extending through said focal volume along said central lens axis (in a generally vertical orientation through the focal plane);
the said excitation light source, for illuminating said focal volume; and
a camera for detecting fluorescence collected from the focal volume of the objective lens in said multiple colour channels;
preferably wherein 90% of the point spread function of the system at the focal plane of the objective lens occupies a camera area corresponding to 49 pixels or fewer,
the method comprising:
(1) said labelling step, involving incubating the sample with fluorescent probe (a) and at least one of fluorescent probes (b) and (c);
(2) said imaging step, involving flowing the sample through said fluidic channel vertically through the focal volume and imaging the sample within the channel using the fluorescence imaging system to produce fluorescence image data for each of the multiple colour channels;
(3) said measurement step, involving detecting the presence of candidate objects in the fluorescence image data which display fluorescence above a threshold, and recording the signal in the multiple colour channels for each candidate object as sample data; and
(4) said characterisation step, involving inputting the sample data into a machine learning classification algorithm to generate result data, wherein the result data includes whether the candidate objects are pathogens and if so, the type of pathogen.

24. A computer-implemented system for identifying pathogens in a sample, the system being configured to:
train a machine learning algorithm to identify one or more pathogens in sample data, using a labelled set of training data relating to a plurality of candidate objects in training fluorescence image data, the labelled set of training data comprising, for each of the plurality of candidate objects, information indicative of signal intensity in each of multiple colour channels in the training fluorescence image data, wherein each of the multiple colour channels corresponds to a respective fluorescent probe;
receive fluorescence image data corresponding to each of the multiple colour channels;
analyse the received fluorescence image data to detect the presence of a candidate object which displays fluorescence above a threshold, and measure the signal intensity in each of the multiple colour channels for the candidate object to provide sample data for the candidate object; and
use the machine learning algorithm to analyse the sample data so as to generate results data, wherein the results data includes whether the candidate objects are pathogens and, if so, the type of pathogen.

25. A computer-implemented system according to claim 24, further configured to:
pool sample data and results data corresponding to samples from multiple patients; and
analyse the pooled data.

26. A kit of parts for carrying out a method according to claim 1, comprising at least two fluorescent probes selected from fluorescent probe (a), fluorescent probe (b) and fluorescent probe (c).
